(19) United States
(12) Patent Application Publication
(10) Pub. No.: US 2026/0245719 A1
(43) Pub. Date: Aug. 20, 2026

(54) A COMPUTER IMPLEMENTED SYSTEM AND METHOD TO ASSIST IN PATIENT CARE DELIVERY, DRIVING VEHICLES, PROVIDING SAFETY, SECURITY, CUSTOMER SUPPORT AND IDENTITY VALIDATION

(71) Applicant: Azad Alamgir Kabir, Biloxi, MS (US)

(72) Inventor: Azad Alamgir Kabir, Biloxi, MS (US)

(21) Appl. No.: 18/707,575

(22) PCT Filed: Dec. 23, 2022
(86) PCT No.: PCT/US2022/053905

§ 371 Date: May 4, 2024

(51) Int. Cl. G16H 40/67; B60W 40/08; B60W 50/14; B60W 60/00; G06F 3/16; G06T 7/70; G06V 40/10; G06V 40/20; G10L 15/26; G16H 10/60; G16H 15/00; G16H 20/00; G16H 50/20; G16H 50/30; G16H 80/00

(52) U.S. Cl. CPC G16H 40/67; B60W 40/08; B60W 50/14; B60W 60/0015; G06F 3/167; G06T 7/70; G06V 40/10; G06V 40/28; G10L 15/26; G16H 10/60; G16H 15/00; G16H 20/00; G16H 50/20; G16H 50/30; G16H 80/00; B60W 2040/0818; B60W 2540/229; B60W 2554/406; G06T 2207/30196

(57) ABSTRACT

A computer implemented system to assist a user includes a cognitive computation module which can speak to, listen to and/or see. The system can assist in patient care delivery, driving, providing security, customer support and identity verification. It may be configured in a processor of the computing device which receives text data input after converting speech into text. The received text data is analysed by the module using an artificial intelligence and/or decision rules modules. The modules may create a response of text output which is converted to speech data using text to speech module and sent to the communication module to speak out. It identifies posture of human body, or the face of a human or a user using computer connected camera and also receives input from other sensors or devices connected to the computer and sends input to the cognitive communication module for analysis and response.

### FIELD OF THE DISCLOSURE

The present disclosure relates to field of a computer implemented system and method to assist in day-to-day daily activity using its camera, speaker, microphone and other sensors which can function like a humanoid robotic assistant which can use all language to communicate. More particularly, it is computer implemented system and method to diagnose a disease or a medical condition to provide treatments, alerts, or assistance, to assist driver or user during driving a vehicle, to provide safety, security to a premises, to provide customer support and validate identity of a user or a human.

### BACKGROUND OF THE DISCLOSURE

Healthcare system capacity is the essential limiting factor in the COVID-19 epidemic response. The assurance of quality of patient care is compromised by the limitations on how many patients any given doctor or nurse practitioner can see, diagnose, triage, and treat optimally with the best standards of care. A cognitive clinical assistant may precisely solve this problem, allowing for rapid diagnosis and triage as well as essential support in treatment. Critically, this cognitive clinical assistant may address not only the needs of the U.S. healthcare system, but also the dire circumstances that most developing country healthcare systems are facing because of a scarcity of trained physicians. Similar to the healthcare system, human driving is already proven to be less safe than automated driving. The reason is lack of attention and multi-tasking while driving. However, the conventional automated driving systems are not able to communicate to driver/user and fails to alert the driver for potential threats or incoming traffic accidents. The conventional automated driving fails to talk (speak or listen) to the human driver and provide instructions and further fails to monitor response to instruction and take over driving or direction of vehicle movements if life threatening events are within the realm. Further, similar to the healthcare system and driving system, home and commercial use Safety, Security and Customer Support System can be very challenging in the 21st century. The current safety and security systems are not able to speak or listen while seeing intruder or the owner/resident of home or business and fails to eliminate false positive alerts. All current system uses professional call centers that are run by human who call the owner or residents to eliminate false positive alerts before notifying the police or takes the necessary steps. Also, inside any business, human security guards are used to monitor continuous video feed and human security and safety officers are deployed to eliminate false positive conditions.

Accordingly, this invention claiming priority form the provisional patent applications submitted to Unites States Patent and Trademark Office (USTPO) as listed below:
1) Application No: 63/300,663—Titled ‘Building a Robot Doctor for Healthcare Delivery and beyond’,
2) Application No: 63/397,552—Titled “‘Building a Robot Doctor for Healthcare Delivery and beyond’,
3) Application No: 63/400,061—Titled: Robotic Safety, Security, and Customer Support System’,
4) Application No: 63/407,788—Titled ‘Building a Robotic Driving Assist Using Computer Vision and Voice Recognition Technology’ and
5) Application No: 63/405,824—Titled ‘Building a Robotic Driving Assist Using Computer Vision and Voice Recognition Technology’.

There exists a need of a system and method which may increase the number of critical patients that doctors can treat, improve drivers' safety, and reducing road traffic accident, improve safety, security, customer support at home and commercial premises and validation of once identity using voice prompts. Further, there exists a need of such system and method which may reduce the number of face-to-face interactions that healthcare providers must have with patients. Further, there exists a need of such a system and method which may reduce operational expenditures for healthcare facilities. Furthermore, there exists a need of such a system and method which may improve patient welfare by reducing wait times and the hassle. Furthermore, there exists a need for such system and method which may communicate to driver/user and alert the driver/user for potential threats or incoming traffic accidents. Furthermore, there exists a need for such system and method which may communicate with the driver/user, monitor response to instruction, and may take over driving if necessary. Furthermore, there exists a need of such system and method which may communicate to the intruder or the owner/resident of home or business and eliminate false positive alerts. Furthermore, there exists a need of such system and method which may replace use of professional call centers calling the owner or residents to eliminate false positive alerts before notifying the police or to take the necessary steps. Furthermore, there exists a need of such system and method which may augment human security guards to monitor continuous video feed and help security and safety officers to eliminate false positive conditions. The computer implemented system that has the ability to speak, or listen and or seeing, can assist elderly population to age at home. The system monitored the user continuously which can be as simple as a computer hanging on the wall or a humanoid robot moving in the floor using automated guided vehicle (AGV). Furthermore, there exists a need of such system and method to validate anyone identity using voice prompts using computer connected sensors, camera, speaker and microphone where user can log into the computer using voice prompt, search internet without using keyboard (using voice prompts), allow users to log on bank account, social media and turn on or turn off any electronic controlled machinery or devices or vehicles using voice prompt.

### SUMMARY OF THE DISCLOSURE

In view of the foregoing disadvantages inherent in the prior art, the general purpose of the present disclosure is to provide a computer implemented system to diagnose a disease and or provide treatments to include all advantages of the prior art, and to overcome the drawbacks inherent in the prior art.

An object of the present disclosure is to increase the number of critical patients that doctors can treat or a nurse can manage in the hospital, nursing home, rehabs, clinics, urgent care center or at home by assisting in clinical care delivery.

Another object of the present disclosure is to create an intelligent remote monitoring system which reduce the need for patient hospitalizations, or number of face-to-face interactions that healthcare providers must have with patients.

Yet another object of the present disclosure is to reduce operational expenditures for healthcare facilities by reducing unnecessary testing, reducing utilization of healthcare resources, reducing length of hospital stay for patients, preventing patient's readmission.

Yet another object of the present disclosure is to improve patient welfare by reducing wait times at healthcare facilities, reduce the hassle of patients travelling to meet the physicians in person and reducing exposure to highly communicable diseases by allowing patients stay at home.

Yet another object of the present disclosure is to provide a system and method which may communicate to driver/user and alert by speaking to the driver/user for potential threats or incoming traffic accidents.

Yet another object of the present disclosure is to provide system and method which may communicate with the driver/user, by listening verbal response to instruction, and may take over driving if necessary.

Yet another object of the present disclosure is to diagnoses patient medical condition and determine that the driver is incapacitated to drive, may take over driving, start auto pilot driving and drive to nearest hospital as well as inform emergency medical services on its way about drivers' medical conditions.

Yet another object of the present disclosure is to provide system and method which may use an artificial intelligence/driving assist algorithm to control the vehicle.

Yet another object of the present disclosure is to advice by seeing surroundings, speaking and listening responses of the driver about changing lanes whether it is safe to change or not. In addition, also advice based on information obtained from surrounding vehicles.

Yet another object of the present disclosure is to provide system and method which may speak and listen while seeing the intruder or the owner/resident of home or business and eliminate false positive alerts.

Yet another object of the present disclosure is to provide system and method which may automate the process of notifying the police or to take the necessary steps when intruder or human fails to verify the security code which might reduce need of professional call centers calling the owner or residents to further reduce the cost of monitoring.

Yet another object of the present disclosure is to provide system and method which may augment human security guards to monitor continuous video feed and assist providing security and safety by eliminate false positive conditions.

Yet another object of the present disclosure is to seeing the customers activity is a super market or any commercial business and speak to the customers if they are attempting to steal any goods. Once identified the not allowed activity inside a commercial business location, the system will speak to the customer and listen to their responses regarding prohibited activity. If issues are not resolved with speaking and listening, the system will connect with police or security guards to intervene.

In view of the above objects, in one aspect, a computer implemented system to diagnose a disease and or provide treatments may include a cognitive computation module configured in a computing device which can speak to, listen to and or see any patient or any user. The cognitive computation module may be configured in a processor of the computing device which receives text data input from a coupled connected communication module after converting speech to text input. The received text data may be analysed by the cognitive computation module using an artificial intelligence and a decision rule module. The cognitive computation module may create a response of text output which may be converted to speech data using text to speech module and then may be send to the communication module to communicate the response.

The cognitive computation module may receive further input from the patient or user using the communication module and the cognitive computation module may analyse all input and find a diagnosis and may further provide treatment recommendation by writing an encounter note for the patient visit.

The cognitive module of the computer implemented system may be coupled/linked with all available data in the electronic medical records which consists of patient's signs, symptoms, laboratory findings, radiological findings, and treatment data.

In one embodiment of the present disclosure, the Artificial intelligence module of the computer implemented system may be coupled with the cognitive computation module with all possible signs, symptoms, laboratory findings, radiological findings, and treatment strategies.

In one embodiment of the present disclosure, the computing device of the computer implemented system may be one of a desktop, a mobile phone, a laptop, a tablet, a wearable device, a robot, etc.

In one embodiment of the present disclosure, the cognitive computation module of the computer implemented system may help live stream the computer video and or patient vital signs or telemetry data (from the connected devices) to a remote location using either a laptop, desktop, tablet, robot, a wearable device, any computing device or a mobile phone.

In one embodiment of the present disclosure, the communication module the computer implemented system may consists of microphone, speaker, any sensor, a camera, and Bluetooth connected devices like blood pressure cuff, pulse oximetry, blood sugar meter, weighing scale, Wi-Fi (wireless fiber) connected devices like telemetry system, other medical devices, cellular connected devices (using 2G, 3G, 4G, or 5G cards) like mobile phone, blood pressure cuff, pulse oximetry, blood sugar meter, weighing scale, or a combination thereof.

In one embodiment of the present disclosure, the camera of the communication module may identify the physical position of or gesture of the patient; and the communication module speak to the patient, listen to patient's response and communicate with a caregiver or provider.

In one embodiment of the present disclosure, the cognitive computation module may help connect to a video call without needing any touch from the user and the video call may be initiated from a remote location using either a laptop, desktop, tablet, robot, a wearable device, any computing device or a mobile phone.

In one embodiment of the present disclosure, the cognitive computation module of the computer implemented system may be connected to the other electronic medical records or data repository, network systems for fetching information, medical data, etc.

In one embodiment of the present disclosure, the cognitive computation module of the computer implemented system may serve as providers rounding assistant by speaking out patient's vitals, laboratory data, radiological data, microbiology data, provider's notes and other data from the electronic medical records.

In one embodiment of the present disclosure, the cognitive computation module of the computer implemented system may serve as abnormal data monitoring system when such data become available in any electronic medical records. The cognitive computation module may speak to the patient, listen to patient's response, and communicate with a caregiver or provider about patient clinical status.

In one another aspect, a method for diagnosing disease and providing treatment may be provided. The method for diagnosing disease and providing treatment may include speaking with patient about patient's symptoms, listening to patients' response and asking further questions based on patients' response; and/or transmitting patient's symptoms data to a cognitive computation module configured in an processor of a computing device, analysing all the symptoms data using the cognitive computation module, providing suitable treatment recommendation for the diagnosed disease; and/or storing the history of patient, diagnosis and treatment data in an encounter note.

In one embodiment of the present disclosure, the computing device may be one of a desktop, a mobile phone, a laptop, a tablet, a wearable device, a robot, etc.

In one embodiment of the present disclosure, the communication with the patient and providing suitable treatment may be done via a communication module. The communication module may include microphone, speaker, sensor, a camera or a combination thereof.

In one embodiment of the present disclosure, the speaking and the listening to the patient complains may include questions and every subsequent question may be based on the analysis of the first/previous question done by the cognitive computation module.

In one embodiment of the present disclosure, the cognitive computation module may be adapted to monitor the patient and generate instructions for the patient or to a caregiver based on physical gesture of the patient.

In one embodiment of the present disclosure, the cognitive computation module may be linked with all available signs, symptoms, laboratory findings, radiological findings and treatment data in the electronic medical records.

In one embodiment of the present disclosure, the server may be adapted to connect to the other servers, networks, systems for fetching information, medical data, etc.

In one embodiment of the present disclosure, the cognitive computational module of the computer implemented system may shut down the computer device, restart the device, or increase or decrease speaker volume when receives voice command or hand gesture without needing input from any keyboard or mouse.

In one embodiment of the present disclosure, the cognitive computational module of the computer implemented system may receive input from the camera and determine patient severity of illness categorized as mild, moderate, and severe.

In one embodiment of the present disclosure, the cognitive computational module of the computer implemented system may write a provider progress note or encounter note which may include chief complaints, history of present illness, review of system and treatments.

In one embodiment of the present disclosure, the cognitive computational module of the computer implemented system may speak to patients and educate providers in delivering cost-effective treatments, reduce unnecessary testing, and/or clinical documentation improvement messages and or any other messages.

In one embodiment of the present disclosure, the cognitive computational module of the computer implemented system may speak to patients and educate patients about almost all common medical symptoms and diagnoses.

In one embodiment of the present disclosure, the cognitive computational module of the computer implemented system may send alert for life threatening patient conditions like Code Blue and Rapid Response for hospitalized patients.

In one embodiment of the present disclosure, the cognitive computational module of the computer implemented system may be remotely controlled by another computer where user can start or stop video calls where remote user has total control of the patient facing computer.

In one embodiment of the present disclosure, the cognitive computational module of the computer implemented system can determine how sick the patient is and determine whether patient needs to be admitted to hospital floor versus intensive care unit or treated as outpatient.

In one embodiment of the present disclosure, the cognitive computational module of the computer implemented system may be adapted to speak or listen in any language and submit the response or transcript of conversation or encounter note in English or any chosen language.

In one embodiment of the present disclosure, the cognitive computational module of the computer implemented system may be adapted to send alert for diagnosis of sepsis or severe sepsis or septic shock.

In one embodiment of the present disclosure, the cognitive computational module of the computer implemented system may be adapted to send alert for any diagnosis or life-threatening diagnosis.

In one embodiment of the present disclosure, the cognitive computational module of the computer implemented system may be adapted to send alert for life threatening patient condition when abnormal or critical data is available.

In one embodiment of the present disclosure, the cognitive computational module of the computer implemented system may be adapted to send alert for diagnosis of sepsis or severe sepsis or septic shock.

In one embodiment of the present disclosure, the cognitive computational module of the computer implemented system may be adapted to send alert for diagnosis of sepsis or severe sepsis or septic shock.

In one embodiment of the present disclosure, the cognitive computational module of the computer implemented system may be adapted to send alert for any diagnosis or life-threatening diagnosis.

In one embodiment of the present disclosure, the cognitive computational module of the computer implemented system may be adapted to provide recommendation about patient visiting emergency room or contacting physicians or nurses for further healthcare.

In one embodiment of the present disclosure, the cognitive computational module of the computer implemented system may be adapted to provide recommendation about visiting emergency room or contacting a physician or a nurse for further healthcare.

In one embodiment of the present disclosure, the cognitive computational module of the computer implemented system may be adapted to notify users about conditions of patients waiting to be seen.

In one embodiment of the present disclosure, the cognitive computational module of the computer implemented system may be adapted to notify users about conditions of patients waiting to be seen.

In another embodiment, the computer implemented system to diagnose a disease and or provide treatments will increase the number of critical patients that doctors can treat: the computer implemented system to diagnose a disease and or provide treatments can achieve this by equipping physicians' assistants and nurse practitioners to identify high-risk patients and diagnose & triage a higher volume of patients effectively without demanding the immediate or elaborate attention of the attending doctors and specialists.

The computer implemented system to diagnose a disease and or provide treatments Reduce the number of face-to-face interactions that healthcare providers must have with patients: the computer implemented system to diagnose a disease and or provide treatments can, through a more efficient and automated triage process, reduce the need for face-to-face interactions between high-risk patients and healthcare providers, thereby reducing exposure and transmission risks and the exhaustion of scarce PPE equipment.

The computer implemented system to diagnose a disease and or provide treatments may reduce operational expenditures for healthcare facilities: Clinical Assist will help increase healthcare access by providing healthcare at a negligible cost through improved allocation of services and reductions in unnecessary treatments and defensive medicine practices, reduce the costs of overburdened healthcare systems in an era when governments and banks are struggling to provide the loans required to keep such businesses afloat; and

The computer implemented system to diagnose a disease and or provide treatments may improve patient welfare by reducing wait times, the hassle (and transmission risk) of physically going to a healthcare center, incorrect diagnoses and treatments due to lack of time and failure to attend to all symptoms and consider all possible diagnoses (as CLINICALASSIST) should reduce the likelihood of mistakes of memory which are frequent among healthcare providers, especially overburdened ones). Finally, we cannot emphasize enough how a platform such as the computer implemented system to diagnose a disease and or provide treatments is desperately needed in health settings in a pandemic crisis in the USA and globally, and in particular, in developing country contexts where healthcare systems are incredibly poorly equipped, physicians poorly trained and inadequately available, a tool such as this could enormously enhance their ability to optimally manage any pandemic.

In one embodiment, the patient may speak about their medical problems with the computer implemented system to diagnose a disease and or provide treatments within its hearing range.

In one embodiment, the computer implemented system to diagnose a disease and or provide treatments (Cognitive computation module) may listen using the Microphone attached with it.

In one embodiment, the computer implemented system to diagnose a disease and or provide treatments may convert the received speech (of patient's) to text data. The converted text data (patient complains or symptoms) may be submitted to the Cognitive computation module (Robotic Clinical Assist) for analysis

In one embodiment, the computer implemented system to diagnose a disease and or provide treatments may utilize cognitive computation algorithm which is artificial intelligence algorithm along with decision rules to analyze the patient symptoms, collects further clinical information and delivering the patient specific treatments.

In one embodiment, the computer implemented system to diagnose a disease and or provide treatments may collect targeted history and clinical information from the patients by sending text messages to communication module's speakerphone which is converted to speech so that speakerphone can utilize the data signal.

In one embodiment, the patient hears the speech from the computer implemented system's speakerphone and responds to questions asked which the computer implemented system to diagnose a disease and or provide treatments can listen and process like human doctor communications.

In one embodiment, the computer implemented system to diagnose a disease and or provide treatments (Cognitive computation module) may see using the camera attached with it.

In one embodiment, the computer implemented system to diagnose a disease and or provide treatments may convert the computer vision data (of patient's) in to computer signal that use cognitive computation modules to monitor patients falling out of the bed.

The converted computer vision signal data about patients' facial recognition data, physician findings, skin lesions, severity of illness and physical findings are submitted to the Cognitive computation module (Cognitive computation module) for analysis and further actions

In one embodiment, the computer implemented system to diagnose a disease and or provide treatments may produce diagnoses and treatment plans as good as human doctors and nurse practitioners and be utilized both in a medical facility and remotely to monitor any patients as safely and quickly as accurately possible. The computer implemented system to diagnose a disease and or provide treatments may help any healthcare system, from rural and urban areas of the United States to healthcare systems across the developing world. The computer implemented system to diagnose a disease and or provide treatments may help 1) reducing the need for face-to-face encounters; 2) reducing the need for personal protective equipment and making telemedicine encounters safer; 3) reducing patient commute times; and 4) reducing volume-related pressure on healthcare systems. The goal is that Cognitive computation module will be used for immediate and effective patient care around the globe during this pandemic, and beyond.

In one embodiment, a computer-implemented system for diagnosing diseases and providing treatments may include a cognitive computation module, a second computing device and a second communication module. The cognitive computation module may be configured in a processor of a first computing device. The first computing device may be having at least a speaker, a microphone, and/or a camera. The second computing device may be coupled with a second communication module which is an internet webpage. The cognitive computation module may receive data inputs from the second computing device and the received data may be analyzed by the cognitive computation module of the first computing device utilizing an artificial intelligence module and/or a decision rule module.

In one embodiment, the first computing device and the second computing device of the computer-implemented system may be interconnected via an internet connection.

In one embodiment, the first computing device and the second computing device of the computer-implemented system may be one of a desktop, a mobile phone, a laptop, a tablet, a wearable device, a robot, etc.

In one embodiment, the first computing device and the second computing device of the computer-implemented system may connect to one another from a remote location.

In one embodiment, the second computing device of the computer-implemented system may be adapted to connect to the first computing device from a remote location for a video or audio call.

In one embodiment, live video feed windows of one or more patients may be displayed on the second computing device from one or more first computing devices of the computer-implemented system.

In one embodiment, each of the video feed window displays respective patient's telemetry data, and or pulse oximetry data and or vital signs data, and or any other data from electronic medical record, and or other remainders data to improve patient safety.

In one embodiment, the conversation between patient and healthcare providers is transcribed which includes at least one of the patient data such as but not limited to history of a patient, physician exam, laboratory data, radiological data, diagnosis data, and treatment data to create AI enhanced encounter notes.

In one embodiment, a computer-implemented system for diagnosing diseases and providing treatments may include a cognitive computation module, a second computing device, second communication module, a third computing device and a third communication module. The cognitive computation module may be configured in a processor of a computer server coupled with internet connection. The second computing device may be coupled with a second communication module which is an internet webpage and the second communication module may include at least a speaker, a microphone, and/or a camera. Further, the third computing device may be coupled with the third communication module which is an internet webpage and the third communication module may include at least a speaker, a microphone, and/or a camera. The cognitive computation module, coupled in a processor of a computer server may receive data input from the second and/or the third computing device and the received data may be analyzed by the cognitive computation module of the computer server utilizing an artificial intelligence module and/or a decision rule module.

In one embodiment, the second computing device and the third computing device of the computer-implemented system may be interconnected via an internet connection.

In one embodiment, the second computing device and the third computing device of the computer-implemented system may be one of a desktop, a mobile phone, a laptop, a tablet, a wearable device, a robot, etc.

In one embodiment, the second computing device and the third computing device of the computer-implemented system may connect to one another from a remote location.

In one embodiment, the second computing device of the computer-implemented system may be is adapted to connect to the third computing device of the computer-implemented system from a remote location for a video or audio call.

In one embodiment, the conversation between patient and healthcare providers is transcribed which includes at least one of the patient data such as but not limited to history of a patient, physician exam, laboratory data, radiological data, diagnosis data, and treatment data to create AI enhanced encounter notes.

The Cognitive computation module contains a complex artificial intelligence system where all medical diagnoses linked with all possible signs, symptoms, and laboratory and radiological findings as well as treatment strategies. In addition, it provides complete decision support for the medical conditions. The cognitive computation module can receive any kind of input and can provide output like an expert doctor.

A computer implemented system to assist a human/user to perform different activities. The computer implemented system to assist a human/user may include a cognitive computation module configured in a computing device which can see using an attached camera, can speak to, listen to a user. The cognitive computation module may be configured in a processor of the computing device which receives text data input from a coupled communication module after converting speech to text input, the received text data may be analyzed by the cognitive computation module which includes an artificial intelligence algorithm and or a decision rule module. The cognitive computation module may create a response of text output which is converted to speech data using text to speech module and then may send to the communication module to communicate the response. At the same time, the computer camera picture or video feed can be analyzed, and real time camera input can be communicated with the cognitive computation module so that many human activities can be performed.

The cognitive computation module may receive further input from the radio-frequency identification (RFID) tags or sensors, Bluetooth connected devices, Wi-Fi connected devices, blood pressure cuff, pulse oximetry devices, telemetry devices, blood sugar meter, ultrasonic sensors, cellular connected devices (using 2G, 3G, 4G or 5G cards), laser sensors, GPS data, IoT devices, and from the user using the communication module and the cognitive computation module may analyses all input and find a best advice to assist the user.

The cognitive computation module can be used to build driving Assist which may reduce road traffic accidents and death by providing active driving assist while driving small or large private or commercial vehicles. Driving Assist can watch and identify incoming traffic threats or potential accidents using computer vision program, voice recognition technology, GPS location technologies and reduce accidents by speaking and listening and watching (or seeing using computer vision signals) and identifying the threats. The system will also use radar, laser sensors or thermal sensors to identify incoming threats and communicate with the driver.

In one embodiment, the computer implemented system may assist a human or user while driving a vehicle, wherein the cognitive computation module may analyses all input and execute the best option to assist the driver/user in the vehicle.

In one embodiment, the computer implemented system may assist a human or user while driving a vehicle. The computer implemented system to assist human or user while driving a vehicle may connect with nearest vehicle if equipped with same technology, communicate incoming threat which surrounds vehicle can't recognized due to its distance from the threats, using voice commands, and computer vision signals: The computer implemented system to assist human or user while driving a vehicle may send signals to surrounding vehicles and alert other drivers to flee the incoming road traffic threats by acceleration or changing lanes or going out of the street to make space for the vehicle behind or in front of them. This technology will help clear the street and create space and reduce further risk of traffic accidents.

The computer implemented system to assist human or user while driving a vehicle may help aircraft pilots while flying and help connect with air traffic control using voice commands, and computer vision signals: Pilot Assist is a standalone device with all features included in each aircraft. As long as connected to electricity and internet, it starts functioning.

The computer implemented system to assist human or user may be used in boat, ships and water vehicles safety for assisting the pilot. The computer implemented system may help safety and security monitoring of water vehicle through providing a robotic personal companion who can speak to the driver or pilot and ask for help, search internet for necessary information using voice commands (without needing to use keyboard or touch screen); and provide assistance if requested.

The computer implemented system may start, turn off, lane change, acceleration, reducing speed and control vehicle using voice commands.

The computer implemented system may be able to execute all the driving needs just from voice commands which will allow driver concentrate to the road traffic situation. Here driver do not need to move their eyes from the road as vehicle will listen to the driver using voice communication while also providing feed backs using computer vision signals. The Driving Assist will keep the vehicle in lane even when driver is inattentive using GPS location technologies and computer vision signals and different sensors.

The computer implemented system may screen for life threatening medical condition of the driver and safely auto pilot driver from off of the street by using emergency driving assist.

The computer implemented system may screen for common medical conditions, screen for psychiatric disease, risk of suicide and life-threatening conditions like seizures and stroke while driving and connect with healthcare providers, emergency medical services, and law enforcement, when necessary, by speaking and listening to driver. Finally, we cannot emphasize enough how a platform such as Driving Assist is desperately needed in the USA and globally, contexts where road traffic deaths ae skyrocketing due to increased traffic accidents.

Using watch or any other IoT devices to control vehicle using voice commands: The computer implemented system may support driver to control their linked vehicle by speaking to the hand watch (or device in hand or pocket) from a distant location as long as both the vehicle and watch (or driver device) is connected to the internet. By using a remote device, driving assist can command the vehicle to start, drive and come to a remote location by auto pilot driving simply by speaking and listening (voice commands).

The computer implemented system may prevent theft of vehicle by recognizing using facial recognition and asking for passwords for new drivers by identifying driver's facial recognition and voice recognition and start speaking and warning the humans about potential violation of the codes of conduct and asks for proof of vehicle ownership. If the customer fails to satisfy the robotic assistant interrogations, then alert the police or security personnel. Auto pilot driving while driver is incapacitated, or driver want to sleep or rest while vehicle is on the street. The driving assist can turn in to auto pilot driving (driving without the help of the human driver) using the input from connected cameras, radar, laser sensor and other IoT devices and GPS location technologies. The auto pilot mode actively keeps vehicles on the street, stay on the lane, while following traffic rules. The vehicle will be able to push brakes, reduce speed, accelerate and change lanes and stop at the traffic lights while on auto pilot mode and notify nearest vehicle about its auto pilots driving status. Most importantly, the auto pilot mode will be able decide independently of any direction or exit strategy from any collision or potential collision by causing minimal damage to all party involved with the motor vehicle collision based on available data. In one embodiment, the cognitive computation module of the computer implemented system may be coupled/linked with environment components like GPS, camera, laser sensor, radar sensor, ultrasonic sensor and surrounding vehicle signals to access data of vehicle surrounding.

The computer implemented system (Robotic Assistant brain) may see using computer vision signals by the using signal input from camera and sensors (laser, radar, and ultrasonic) attached with it.

The computer implemented system (Robotic Assistant brain) may process GPS data, along with all signals from cameras, RFID tags, Bluetooth devices, Wi-Fi devices, Cellular devices, microphone, speaker and assist driving or help with auto-pilot driving.

The computer implemented system use Computer vision technology and converts the received computer vision data into signals differentiate between human, animals, vehicle, distance of nearby objects, activity nearby vehicles and anticipate other vehicles moves based on pattern of their driving, assess risks based on surroundings and activate voice prompts to assist the driver.

The computer implemented system may turn on or turn off vehicles using voice recognition technologies, where vehicle can speak and listen, by using voice commands when provided passwords or security information's.

The computer implemented system may speak with humans when spotted in the range and ask for password and listen to response to disable alerts.

The computer implemented system (cognitive computation module) listens using the Microphone connected with it.

The computer implemented system may convert the received speech (of driver) to text data.

The converted text data (drivers' response) are submitted to the computer implemented system's central processing unit (Robotic Algorithm) for analysis.

The computer implemented system may utilize cognitive computation algorithm which is artificial intelligence algorithm along with decision rules to analyze the human response, collects further information by listening or from cameras or sensors and delivering the specific assistance.

The computer implemented system may collect targeted information from the customer/resident by sending text messages to Robot speakerphone which is converted to speech so that speakerphone can utilize the data signal.

The driver hears the speech from the computer implemented system speakerphone and responds to question asked which Robotic Driving Assist can listen and process like human security or customer support assistant.

The computer implemented system may connect with nearest vehicles and communicate road traffic information and risk of collision threats and alert the nearest vehicles drivers using voice commands to move out of the way form vehicle in front or behind to create space for vehicle in jeopardy.

The computer implemented system may be able to turn on auto pilot driving mode without the help of the human driver and safely drive the vehicle when requested or in a time of emergency using GPS, camera, laser sensor, radar sensor, ultrasonic sensor, Bluetooth sensor, RFID tags, Bluetooth devices, Wi-Fi devices, Cellular connected devices and surrounding vehicle signals.

In one embodiment, the Artificial intelligence module/driving assist algorithm of the computer implemented system may couple with the cognitive computation module with all possible road security and safety related rules and regulation data.

In one embodiment, the computing device of the computer implemented system may be one of a mobile phone, a laptop, a tablet, a wearable device, a robot, personal digital assistant fitted in the vehicle, etc.

In one embodiment, the communication module of the computer implemented system may consist of microphone, speaker, any sensor, a camera or a combination thereof.

In one embodiment, the camera of the communication module identifies the medical condition of the driver body image analysis, facial analysis, physical position of or gesture of the driver/user; and the communication module may speak to the driver/user, listen to driver's/user's response and connect with an emergency number.

In one embodiment, the cognitive computation module of the computer implemented system may be connected to a data repository, network systems for fetching information of traffic rules, etc.

In one embodiment, the cognitive computation module of the computer implemented system may serve as surrounding assistant by speaking out vehicle data, surrounding vehicle data, road condition, traffic condition and other data from the electronic records.

In one embodiment, the cognitive computation module of the computer implemented system serve as abnormal data monitoring system when such data become available in any electronic records and the cognitive computation module may speak to the driver/user, listen to driver's/user's response, and connect with an emergency number about to inform driver's/user's status.

In one embodiment, the cognitive computation module of the computer implemented system may determine if the user or driver is medically incapacitated, and then activates auto pilot driving mode.

In one embodiment, the cognitive computation module of the computer implemented system may determine if street is comparatively empty or less traffic and speaks to the user/driver to activate auto pilot driving mode.

In one embodiment, the cognitive computation module of the computer implemented system may speak to the driver to change lane or take an exit or come to complete stop.

In one embodiment, the cognitive computation module of the computer implemented system may speak to the driver before taking over the driving as auto pilot mode.

In one embodiment, the cognitive computation module of the computer implemented system may speak to the driver to place hands on the steering wheel.

In one embodiment, the cognitive computation module of the computer implemented system may speak to the driver to verify password upon user/drivers' entry into the vehicle and the cognitive computation module validates the password if correct then turn on ignition.

In one embodiment, the driver/user voice may command to drive to a specific destination, turn on ignition, increase or decrease volume, increase or decrease temperature, initiate a phone call, initiate a video call, search nearest restaurant, search any location, manage the personal calendar, read recent emails, messages, or post in social media.

In one embodiment, the cognitive computation module of the computer implemented system may speak to any human who is trying to open the car door or come close to the vehicle to provide entry password and the cognitive computation module validates the password and if correct then allows the human to enter inside the vehicle.

In one embodiment, the cognitive computation module of the computer implemented system may report to the emergency number if incorrect password is provided.

In one another aspect, a computer implemented system to provide security, safety and customer support in premises may be provided. The computer implemented system to provide security, safety and customer support in premises may include a cognitive computation module configured in a computing device which can speak to, listen to and/or see a user. The cognitive computation module may be configured in a processor of the computing device which receives text data input from a coupled communication module after converting speech to text input, the received text data may be analyzed by the cognitive computation module using an artificial intelligence/Safety, Security and Customer Assist Algorithm and a decision rules module. The cognitive computation module may create a response of text output which is converted to speech data using text to speech module and then send to the communication module to communicate the response.

The cognitive computation module may receive further input from the user using the communication module and the cognitive computation module analyses all input and provide security, safety and customer support in a premises.

The computer implemented system to provide security, safety and customer support in a premises may reduce the cost for home and commercial safety and security as it does not need a professional monitoring center. Safety, Security and Customer Assist can watch and identify any intruder, thief, bugle and residents or human using computer vision program and voice recognition technology and reduce professional monitoring station costs and eliminating the false positive by speaking and listening and watching (or seeing using computer vision signals) and identifying the residents or intruder or humans or thief's as well as also identifying one using facial recognition. The high-quality triage done by Robotic Assist can deliver accuracy as if there is a live 24×7 monitoring of the home or business is done by robots.

The computer implemented system to provide security, safety and customer support in a premises may Reduce the hassle of implemented and open and plug in system: The computer implemented system to provide security, safety and customer support in a premises may be a standalone device with all features included in each device. As long as connected to electricity and internet, it starts functioning. Instead of setting it up in each door and window, the system works using computer camera and spots human movements or presence and can separate non-human objects, cart, basket, clothes, dresses, and other objects from humans using computer vison program. This system does not need to monitor windows or doors opening rather strategically placing devices the house or business high traffic and strategic locations and outside the business locations so that all key access point is monitored to ensure safety and security of the residents and business.

The computer implemented system to provide security, safety and customer support in a premises may reduce expenditures for elderly household for safety, security and personal assist: The computer implemented system to provide security, safety and customer support in a premises may help safety and security monitoring of lonely adults at a negligible cost through providing a robotic personal companion who can speak to the device and ask for help, search internet for necessary information using voice commands (without needing to use keyboard or touch screen); and provide assistance if requested, and improve welfare by improving psychological and social support

The computer implemented system to provide security, safety and customer support in a premises may screen for common medical conditions, screen for psychiatric disease, risk of suicide and connect with healthcare providers, when necessary, by speaking and listening to residents. Finally, we cannot emphasize enough how a platform such as the computer implemented system to provide security, safety and customer support in a premises may be desperately needed in the USA and globally, and in particular, in developing country contexts where buildings are incredibly poorly equipped.

The computer implemented system to provide security, safety and customer support in a premises may improve customer support and automated checkout both retail and whole sale stores: The computer implemented system to provide security, safety and customer support in a premises may support customers to check out, help with self-check-out, receive credit cards payments and provide instruction for self-check outs, by watching, speaking and listening to customers.

The computer implemented system to provide security, safety and customer support in a premises may prevent theft in retail and commercial wholesale locations by identifying certain goods placed inside human clothing's and dresses or hidden location outside the cart and start speaking and warning the humans about potential violation of the codes of conduct and asks for rationale for certain actions. If the customer fails to satisfy the robotic assistant interrogations, then alert the police or security personnel.

The computer implemented system to provide security, safety and customer support in a premises may (Robotic Assistant brain) can see using computer vision signals by the camera attached with it.

The cognitive computation module uses computer vision technology and converts the received computer vision data in to signals differentiate between burglary, theft, human movements, fall, and different risky postures and positions and activate voice prompts to rule out false positive conditions.

The computer implemented system to provide security, safety and customer support in a premises may turn on or turn off using voice recognition technologies by voice commands when provided passwords or security information's.

The computer implemented system to provide security, safety and customer support in a premises may speak with humans when spotted in the range and ask for password to disable alerts.

The Cognitive computation module (Robotic Assistant brain) listens using the Microphone attached with it.

The cognitive computation module converts the received speech (of Resident's) to text data.

The converted text data (Resident response) are submitted to the computer implemented system to provide security, safety and customer support in a premises may within its hearing range (Robotic Algorithm) for analysis.

The computer implemented system to provide security, safety and customer support in a premises may utilize cognitive computation algorithm which is artificial intelligence algorithm along with decision rules to analyze the human response, collects further information and delivering the specific assistance.

The computer implemented system to provide security, safety and customer support in a premises may collect targeted information from the customer/resident by sending text messages to Robot speakerphone which is converted to speech so that speakerphone can utilize the data signal.

The residents/customers hear the speech from the computer implemented system to provide security, safety and customer support in a premises may speakerphone and responds to question asked which Robotic The computer implemented system to provide security, safety and customer support in a premises may listen and process like human security or customer support assistant.

In summary, the computer implemented system to provide security, safety and customer support in a premises program lies in the computer which converts a traditional computer that has a camera, microphone or speaker into a security and safety companion that can provide monitor home and commercial property from intruder, burglary, theft, fire, flood, or any natural disasters and provide companionship to elderly. The device also can be connected to all other IoT devices and sensor and control all other IoT devices and convert the home or business into a smart voice-controlled homes or business.

In one embodiment, the cognitive computation module of the computer implemented system may be coupled/linked with premises environment components like GPS, camera, speakerphone, microphone, and sensors as well as API connection with other IoT devices to access data of the premises.

In one embodiment, the Artificial intelligence module/Safety, Security and Customer Assist Algorithm of the computer implemented system may be coupled with the cognitive computation module with all possible security and safety related situations, with all possible questions so that false positive situations are eliminated just by speaking and listening to answers to the questions in addition to using facial recognition data or camera input.

In one embodiment, the possible security and safety related situations is intrusion, burglary, theft, fire, flood, or any natural disasters.

In one embodiment, the computing device of the computer implemented system may be one of a mobile phone, a laptop, a tablet, a wearable device, a robot, a personal digital assistant, etc.

In one embodiment, the communication module of the computer implemented system may be consist of microphone, speaker, any sensor, a camera or a combination thereof.

In one embodiment, the camera of the communication module identifies the possible situation of intruder, burglary, theft, fire, flood, or any natural disasters and the communication module speak to the user, listen to user's response and/or connect with an emergency number.

In one embodiment, the cognitive computation module of the computer implemented system may be connected to a data repository, network systems for fetching information of pattern of intrusion, burglary, theft, fire, flood, or any natural disasters, etc.

In one embodiment, the cognitive computation module of the computer implemented system may be serve as surrounding assistant by speaking out premises data, premises surrounding vehicle data and other data from the electronic records.

In one embodiment, the cognitive computation module of the computer implemented system may be serve as abnormal data monitoring system when such data become available in any electronic records and the cognitive computation module may speak to the user, listen to user's response, and/or connect with an emergency number about to inform user's status.

Further, the Cognitive computation module of the computer implemented system may include a combination of automated chat bot function to process (Cognitive computation module Algorithm located in the computer) the received information and collect patient history using the algorithm that mimics physician's thinking process using voice technology as input and output (instead of keyboard, mouse or touch screen computer or any computer screen) which includes speech to text and text to speech algorithm. In addition, computer camera receives input from the patient and computer can speak and ask more questions from the patients and incorporate those input data in the assessment and plan of the decision support. Traditional chat-bots depends on decision tree algorithm which fails to address acute and chronic medical problems with high complexity. But Cognitive computation module provides an exact physician replica by mimicking physician workflow (Ai Algorithm) to solve all medical problems. The underlying decision-making rules to generate diagnosis are unique and use the laws of diagnostic thinking. When a user locks on the generated differential diagnosis, based on spoken symptoms, the artificial intelligence modules and or decision rules modules shows diagnostic confirmation pathways for each diagnosis. Based on patient clinical scenarios, the AI then confirms or denies diagnosis and provides specific treatment strategies. Finally, it delivers providers encounter note which can be saved into the electronic medical record. Thus, it helps with finding patient diagnostics, confirming the most likely diagnosis, providing treatment decisions, and writing an encounter note as if it is a digital physician assistant. Holistically, it can function as a physician.

In addition, robotic assistant can validate one's identity using facial recognition and confirming the personal identity further by speaking and listening to password provided by the users. Such two stage validation will allow user positively identify and assist user with any internet search (performing any task of searching internet and providing answers), working like a personal secretary (scheduling meeting, making phone calls, sending messages, check emails, check face book, twitter, respond using voice to emails and Facebook or any social media, reminding appointments, helping find lost objects, helping cook, helping with house hold task), making any kind of machine automated and make them run using voice command, can connect to any website and communicate with voice commands, connect to any software and make it run using voice commands, provide a platform for all websites and software so that any website or software or social medial can customize their product and run by voice commands only, and help with any or all activity of human being like banking, money management and commercial transaction. Finally, Identification Assist can connect with all the IoT and convert any building into a smart home or business where users face and voice together confirms a personal identity.

This together with the other aspects of the present disclosure, along with the various features of novelty that characterize the present disclosure, is pointed out with particularity in the claims annexed hereto and forms a part of the present disclosure. For a better understanding of the present disclosure, its operating advantages, and the specified object attained by its uses, reference should be made to the accompanying drawings and descriptive matter in which there are illustrated exemplary embodiments of the present disclosure.

### BRIEF DESCRIPTION OF THE DRAWINGS

The advantages and features of the present disclosure will become better understood with reference to the following detailed description taken in conjunction with the accompanying drawing, in which:

FIG. 1 illustrates a block diagram of a system environment to diagnose a disease and/or provide treatments, in accordance with an exemplary embodiment of the present disclosure;

FIG. 2 illustrates a block diagram of a computer implemented system to diagnose a disease and/or provide treatments, in accordance with an exemplary embodiment of the present disclosure;

FIGS. 3 and 4 illustrates an exemplary diagram of a computer implemented system to diagnose a disease and/or provide treatments, in accordance with an exemplary embodiment of the present disclosure;

FIG. 5 illustrates a flow diagram of the computer implemented method to diagnose a disease and/or provide treatments, in accordance with an exemplary embodiment of the present disclosure;

FIG. 6 illustrates a block diagram of a system environment to assist a human/user while driving a vehicle, in accordance with an exemplary embodiment of the present disclosure;

FIG. 7 illustrates a block diagram of a computer implemented system to assist a human/user while driving a vehicle, in accordance with an exemplary embodiment of the present disclosure;

FIG. 8 to FIG. 11 illustrates series of diagrams of a computer implemented system to assist a human/user while driving a vehicle explaining how spoken conversation works between the driver and the vehicle, in accordance with an exemplary embodiment of the present disclosure;

FIG. 12 illustrates a block diagram of a computer implemented system to provide security, safety and customer support in a premises, in accordance with an exemplary embodiment of the present disclosure;

FIG. 13 illustrates a block diagram of a computer implemented system standing in the patient hospital room facing the patient to provide security, safety and customer support in a premises, in accordance with an exemplary embodiment of the present disclosure;

FIG. 14 illustrates a block diagram of a computer implemented system where telemedicine visit initiated by the doctor, where doctor has the control of the patient facing computer implemented system, where patient does not need to touch or click to accept the telemedicine visit link, in accordance with an exemplary embodiment of the present disclosure;

FIG. 15 shows a computer implemented system showing monitoring patient and speaking to the patient when there was an abnormal finding in the patients' vital signs such as pulse oximetry data, blood pressure data, and telemetry data, in accordance with an exemplary embodiment of the present disclosure;

FIG. 16 shows live video feed windows of one or more patients displayed on the second computing device from one or more first computing devices of the computer-implemented system, in accordance with an exemplary embodiment of the present disclosure;

FIG. 17 shows a physician rounding on patient inside a hospital where the physician asking the computer implemented system that what is the last sets of vitals and the computer implemented system answering the question collecting the data from the electronic medical record where it works as medical assistant improving physicians work flow, in accordance with an exemplary embodiment of the present disclosure;

FIG. 18 shows a physician rounding on patient inside a hospital where the physician asking the patient questions related to the patient medical problem and the patient answering them while the computer implemented system on the background transcribing them and subsequently converting them into a physician encounter note for the visit, in accordance with an exemplary embodiment of the present disclosure;

FIG. 19 shows an example of a patient physician conversation during the patient rounding and the computer implemented system transcribing them exactly as it is and subsequently transferring them to a website for physician view and modification using a edit button (to add modify and delete unnecessary component), in accordance with an exemplary embodiment of the present disclosure;

FIG. 20 shows an example of creation of a physician encounter note from the patient physician conversation during the patient rounding (which was transcribed by the computer implemented system) and converted to physician progress notes or encounter notes by using Ai algorithm, designed to improve physician workflow and reduce work load, in accordance with an exemplary embodiment of the present disclosure; and

FIG. 21 shows the operational pathways and elements of a cognitive computation system, in accordance with an exemplary embodiment of the present disclosure.

FIG. 22 shows installation of patient facing cognitive computation module on a hospital patient room, in accordance with an exemplary embodiment of the present invention.

FIG. 23 shows a humanoid robot rescuing a human from a hazardous wildfire situation. The scene captures the urgency and bravery of humanoid robot amidst the chaos of the flames.

FIG. 24 shows a humanoid robot rescuing a soldier from an active bullet fire situation on a battlefield. The scene captures the intensity and heroism of a humanoid robot amidst the chaos.

Like reference numerals refer to like parts throughout the description of several views of the drawing.

### DESCRIPTION OF THE DISCLOSURE

The exemplary embodiments described herein detail for illustrative purposes are subject to many variations in implementation. The present disclosure provides a computer implemented system and method to diagnose a disease and/or provide treatments. It should be emphasized, however, that the present disclosure is not limited to the computer implemented system and method to diagnose a disease and/or provide treatments. It is understood that various omissions and substitutions of equivalents are contemplated as circumstances may suggest or render expedient, but these are intended to cover the application or implementation without departing from the spirit or scope of the present disclosure.

The terms “a” and “an” herein do not denote a limitation of quantity, but rather denote the presence of at least one of the referenced items.

The terms “having”, “comprising”, “including”, and variations thereof signify the presence of a component.

The disclosure provides a computer implemented system to diagnose a disease and/or provide treatments, to assist a driver/user while driving a vehicle and to provide security, safety and customer support in a premises. The computer implemented system may include a cognitive computation module and a connected communication module. The cognitive computation module may be configured in a processor of the computing device which can speak to, listen to and or see any patient, driver and/or user. Further, the cognitive computation module may receive text data input from the connected communication module after converting speech to text input. The received text data may be analyzed by the cognitive computation module using an artificial intelligence/Safety, Security and Customer Assist Algorithm/driving assist algorithm and a decision rules module. The artificial intelligence/Safety, Security and Customer Assist Algorithm/driving assist algorithm and a decision rules module may be coupled with the cognitive computation module. The cognitive computation module creates a response of text output which is converted to speech data using text to speech module and then send to the communication module to communicate the response. The cognitive computation module receives further input from the patient, driver and/or user using the communication module. The cognitive computation module analyses all input and find a diagnosis and provide treatment recommendation by writing an encounter note for the patient, provide assistance to the driver/user while driving a vehicle and/or to provide security, safety and customer support in a premises.

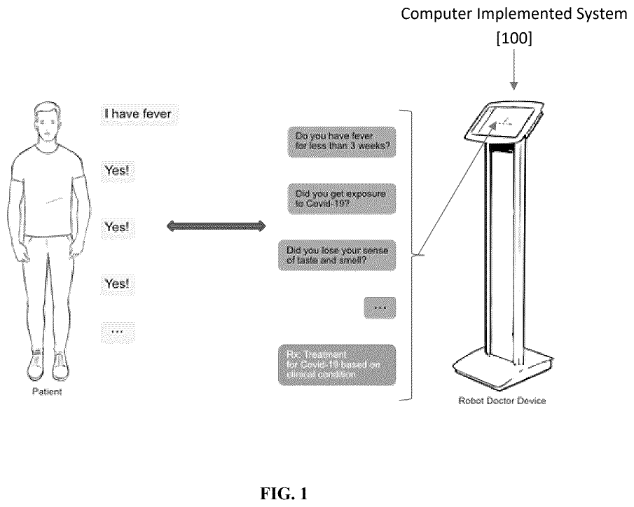

Referring now to FIG. 1, a block diagram of a system environment is shown, in accordance with an exemplary embodiment of the present disclosure. As shown in FIG. 1, in one embodiment, the patient and the computer implemented system to diagnose a disease and/or provide treatments are present. The patient may be present to report a health problem and expect diagnosis and treatment of the disease after diagnosis. The computer implemented system to diagnose a disease and/or provide treatments may attend the patient and may be adapted to solve the problem of the patient. There may be no requirement of any third party for the disease diagnosis and for the treatment of the patient. The third party may include human, machine or a combination thereof.

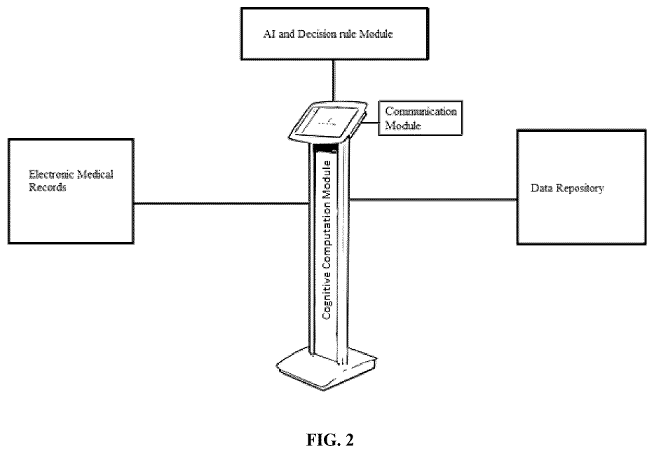

Referring now to FIG. 2, a block diagram of a computer implemented system is shown, in accordance with an exemplary embodiment of the present disclosure. The computer implemented system may include a cognitive computation module, a communication module, an Artificial intelligence module with a decision rules modules and a server. The cognitive computation module may be coupled to the communication module, the Artificial intelligence module, the decision rules modules and the server. The cognitive computation module may be configured in a processor of the computing device. Further, the cognitive computation module of the computer implemented system to diagnose a disease and/or provide treatment may be linked with all available data in the electronic medical records which consists of patient's signs, symptoms, laboratory findings, radiological findings, and treatment data. The cognitive computation module may access the available data as and when required to diagnose a disease and/or provide treatment to the patient. Further, the cognitive computation module of the computer implemented system may be coupled to the other electronic medical records or data repository, network systems for fetching information, medical data, etc.

The Artificial intelligence module of the computer implemented system coupled with the cognitive computation module may contain all possible signs, symptoms, laboratory findings, radiological findings, and treatment strategies. The Artificial intelligence module may assist the cognitive computation module as and when required to diagnose a disease and/or provide treatment to the patient.

The computing device of the computer implemented system to diagnose a disease and/or provide treatment may be one of a desktop, a mobile phone, a laptop, a tablet, a wearable device, a robot and the like. Further, the communication module of the computer implemented system to diagnose a disease and/or provide treatment may include microphone, speaker, any sensor, a camera or a combination thereof. The microphone may be used to receive the speech whenever the patient speaks. The speaker may be used by the computer implemented system to communicate with the patient like for example to ask questions. The camera of the computer implemented system may be used to identify the physical position of or gesture of the patient. In one embodiment of the disclosure, the communication module speaks to the patient, listen to patient's response and communicate with a caregiver or provider.

The cognitive computation module of the computer implemented system may serve as providers rounding assistant by speaking out patient's vitals, laboratory data, radiological data, microbiology data, providers notes and other data from the electronic medical records.

The cognitive computation module may be configured in a processor of the computing device which can speak to, listen to and or see any patient or any user. Further, the cognitive computation module may receive text data input from the communication module after converting speech to text input. The received text data may be analyzed by the cognitive computation module using the artificial intelligence modules and or a decision rule module. The cognitive computation module creates a response of text output which may be converted to speech data using text to speech module and may send the speech data to the communication module to communicate the response. The cognitive computation module may receive further input from the patient or user using the communication module. The cognitive computation module may analyse all input and find a diagnosis and provide treatment recommendation by writing an encounter note for the patient.

The cognitive computation module of the computer implemented system may serve as abnormal data monitoring system when such data become available in any electronic medical records. The cognitive computation module may speak to the patient, listen to patient's response, and communicate with a caregiver or provider about patient clinical status.

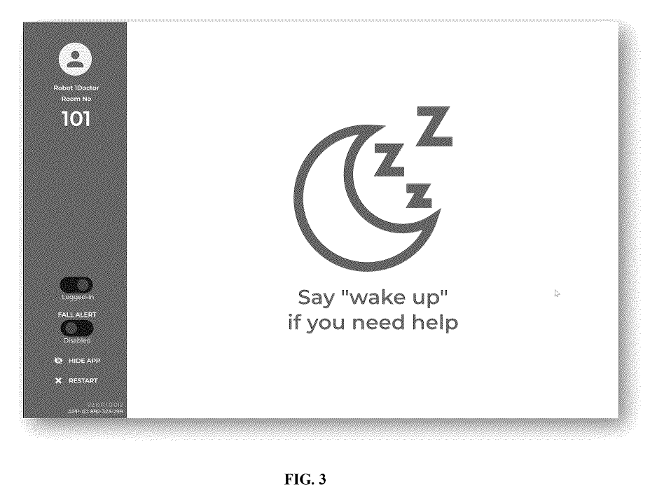

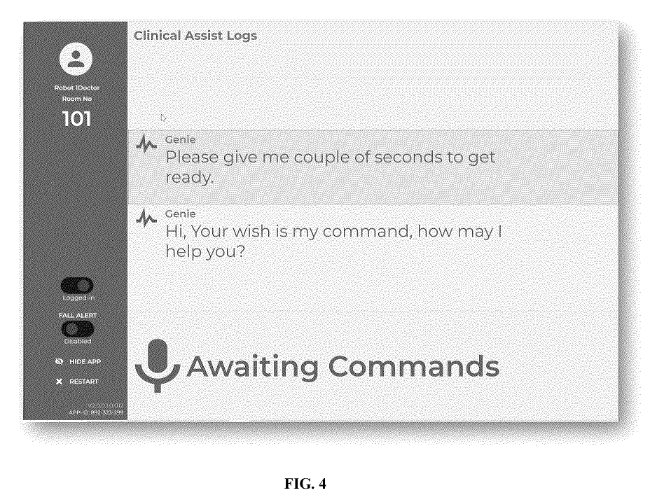

Referring now to FIG. 3, a voice command example of a computer implemented system is shown, in accordance with an exemplary embodiment of the present disclosure. FIG. 3 will now be explained in conjunction with FIG. 1 to FIG. 2. The computer implemented system is installed in room no. 101. The room no. 101 may have a patient and the computer implemented system may be capable of receiving the voice command from the patient. The computer implemented system may be active to provide assistance to the patient upon receiving the voice command from the patient or attendant. Similarly, in FIG. 4, the computer implemented system is in active mode and may be awaiting the voice command from the patient or the attendant.

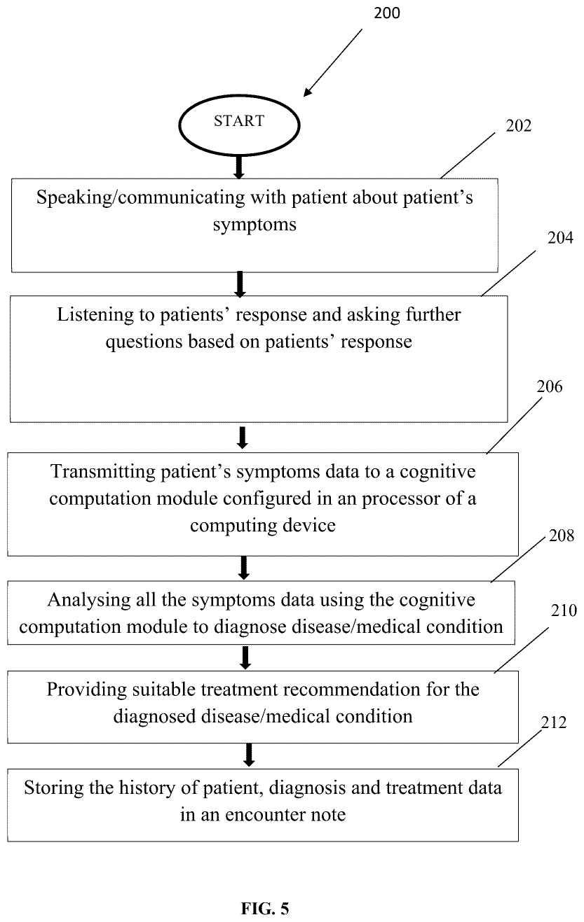

Referring now to FIG. 5, a flow diagram of a computer implemented method is shown, in accordance with an exemplary embodiment of the present disclosure. FIG. 5 will now be explained in conjunction with FIG. 1 to FIG. 4. FIG. 5 shows a flow diagram of a computer implemented method 100 to diagnose a disease and/or provide treatment. The method 200 starts at step 202, the method 200 may include speaking/communicating with patient about patient's symptoms. The communication may be between the patient and the computer implemented system. In one embodiment, the cognitive computation module may receive text data input from the connected communication module after converting speech to text input. The speech may be from the patient and received by the communication module. In one embodiment, the communication module may include microphone, speaker, sensor, a camera or a combination thereof. Thereafter, the method 200 flows to step 204. At step 204, the method 200 may include listening to patient's response and asking further questions based on patient's response. In one embodiment, the further/subsequent questions may be every subsequent question is based on the analysis of the first/previous question done by the cognitive computation module. Further, at step 206, the method 200 may include transmitting patient's symptoms data to a cognitive computation module configured in a processor of a computing device. In one embodiment, the computing device is one of a desktop, a mobile phone, a laptop, a tablet, a wearable device, a robot and the like.

Further at step 208, the method 200 may include analyzing all the symptoms data using the cognitive computation module to diagnose disease/medical condition. In one embodiment, the cognitive computation module may be coupled/linked with all available signs, symptoms, laboratory findings, radiological findings and treatment data in the electronic medical records. The cognitive computation module may be coupled with the Artificial intelligence and decision rule module. The Artificial intelligence and decision rule module may assist in analysing all the symptoms data using the cognitive computation module to diagnose the disease/medical condition. Thereafter, the method 200 flows to step 210, at step 210 the method 200 may include providing suitable treatment recommendation for the diagnosed disease/medical condition. Further, at step 212, the method 200 may include storing the history of patient, diagnosis and treatment data in an encounter note.

The method 200 for diagnosing disease and providing treatment of the cognitive computation module may be adapted to monitor the patient and generate instructions for the patient or to a caregiver based on physical gesture of the patient.

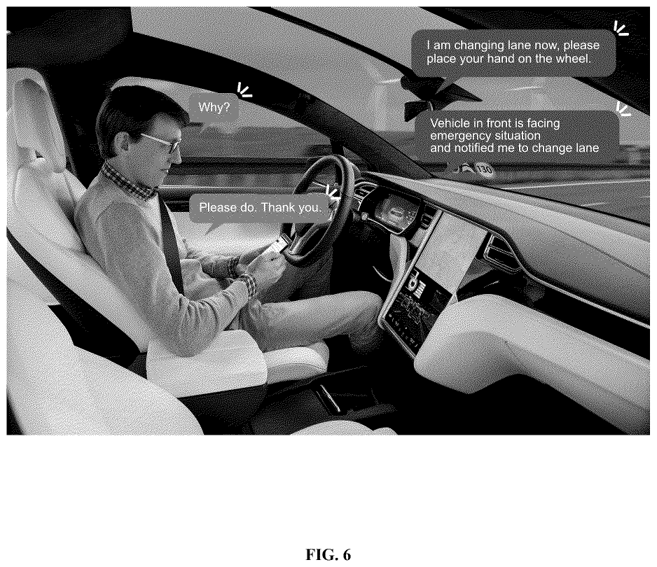

Referring now to FIG. 6, a block diagram of a system environment is shown, in accordance with an exemplary embodiment of the present disclosure. As shown in FIG. 6, in one embodiment, a driver/user and the computer implemented system to assist a driver/user while driving a vehicle are present. The driver/user may be present in the vehicle to drive the vehicle and expect assistance from the computer implemented system. The computer implemented system to assist a driver/user while driving a vehicle may attend/recognize the driver/user and may be adapted to interact with the driver/user in order to assist the driver/user while driving a vehicle. There may be no requirement of any third party for the assistance while driving a vehicle. The third party may include human, machine or a combination thereof.

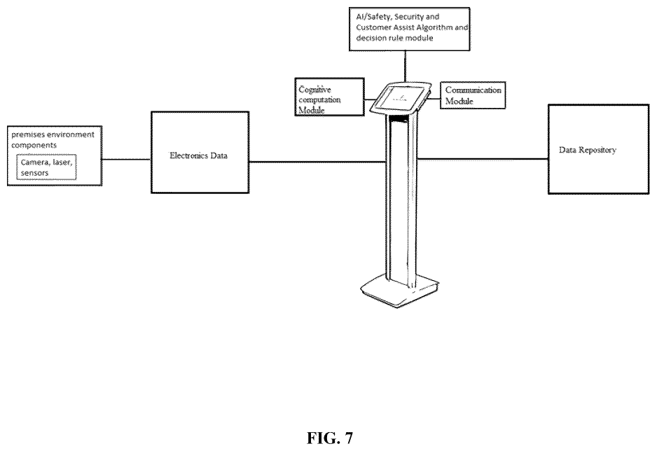

Referring now to FIG. 7, a block diagram of a computer implemented system is shown, in accordance with an exemplary embodiment of the present disclosure. FIG. 7 will now be explained in conjunction with FIG. 6. The computer implemented system may include a cognitive computation module, a communication module, an Artificial intelligence module/driving assist algorithm with a decision rules module and a server. The cognitive computation module may be coupled to the communication module, the Artificial intelligence module/driving assist algorithm, the decision rules modules and the server. The cognitive computation module may be configured in a processor of the computing device. Further, the cognitive computation module of the computer implemented system to assist the driver/user while driving a vehicle may be linked with electronic data which consists of data of vehicle surrounding. The cognitive computation module may access the electronic data as and when required to assist a driver/user while driving a vehicle. The electronic data may consist of data from the environment components like GPS, camera, laser sensor, radar sensor, ultrasonic sensor and the like. The environment components may be responsible of capturing data outside the vehicle or vehicle surrounding. Furthermore, the cognitive computation module of the computer implemented system may be coupled to the other electronic records or data repository, network systems for fetching information of traffic rules, etc.

The Driving Assist is built by connecting a computer (with or without a computer screen) with a computer camera, speakerphone, microphone, and sensors (e.g. laser sensors, radar sensor, ultrasonic sensor) as well as API connection with other IoT devices (and sensors). The Driving Assist Brain Algorithm is uploaded in the computer device. Once the Robotic Assistant algorithm is implemented in the computer, it will receive computer vision signals from the camera, activate the voice recognition system, where voice will be used as a input using the microphone (eliminates the need to use the computer screen or keyboard for data entry) and convert speech into text, then provide the text to the Driving Assist algorithm (brain) where text (data of Resident response) are analyzed, and the output is produced using the Robotic Assistant algorithm were the output is converted to speech and spoken by the connected speaker (e.g. Voice recognition technology). In addition, the computer camera vision data is converted into signal data which is processed in the Robotic Assistant brain and provided input to the Robotic Assistant brain and processed. After signal data are analyzed, the output is produced using the Robotic Assistant algorithm where the output is converted to speech and spoken by the connected speaker. This cycle is repeated. It may or may not need human response voice as an input to process computer vision signal data. However, if it does, then it can use the microphone and convert speech into text, then provide the text to the Robotic Assistant algorithm (brain) where text (data or human response) is analyzed, and the output is produced using the Robotic Assistant algorithm where the output is converted to speech and spoken by the connected speaker. The system can be further connected with all other IoT devices via API or messaging system where whole home or business can be converted into a smart home or business.

The Artificial intelligence module of the computer implemented system coupled with the cognitive computation module may contain all possible road security and safety related rules and regulation data. The Artificial intelligence module may assist the cognitive computation module as and when required to assist a driver/user while driving a vehicle.

The computing device of the computer implemented system to assist a driver/user while driving a vehicle may be one of a desktop, a mobile phone, a laptop, a tablet, a wearable device, a robot and personal digital assistant fitted in the vehicle the like. Further, the communication module of the computer implemented system to assist a driver/user while driving a vehicle may include microphone, speaker, any sensor, a camera or a combination thereof. The communication module of the computer implemented system may be responsible for capturing data inside the vehicle. The microphone may be used to receive the speech whenever the driver/user speaks. The speaker may be used by the computer implemented system to communicate with the driver/user like for example to ask questions. The camera of the computer implemented system may be used to identify the physical position of or gesture of the driver/user. In one embodiment of the disclosure, the communication module may speak to the user/driver, listen to driver's/user's response and communicate with an emergency number.

The cognitive computation module of the computer implemented system may serves as surrounding assistant by speaking out vehicle data, surrounding vehicle data, road condition, traffic condition and other data from the electronic records.

The cognitive computation module may be configured in a processor of the computing device which can speak to, listen to and/or see the driver/user. Further, the cognitive computation module may receive text data input from the communication module after converting speech to text input. The received text data may be analyzed by the cognitive computation module using the artificial intelligence and a decision rules module. The cognitive computation module creates a response of text output which may be converted to speech data using text to speech module and may send the speech data to the communication module to communicate the response. The cognitive computation module may receive further input from the driver/user using the communication module. The cognitive computation module may analyses all input and to assist a driver/user while driving a vehicle.

The cognitive computation module of the computer implemented system may serve as abnormal data monitoring system when such data become available in any electronic records. The cognitive computation module may speak to the driver/user, listen to driver's/user's response, and connect with an emergency number about to inform driver's/user's status. In one embodiment, the computer implemented system to assist a human/user while driving a vehicle may also be referred in as driving assist.

Driving Assist is using a computer connected with a camera, speaker, microphone and laser sensors (and other sensors) that communicate with each other using voice prompts or voice commands (e.g., voice recognition technology). The Robotic Driving Assistant is a device without keyboard, mouse and with or without screen that can effectively communicate with human drivers by using voice commands and assist real time to drive a motor vehicle in the street. The system can greet the human after identifying using facial recognition and disable the vehicle ignition if driver is not recognized. Vehicle can be started, turned off, speed up, or execute a break using voice commands. The current invention will talk to the human driver and provide instructions, monitor response to instruction, and take over driving or direction of vehicle movements if life threatening events are within the realm. This system will assist in auto pilot driving where human driver will remain in charge and improve as complete auto pilot driving showing increasing risk of failure. The road traffic conditions will be activity monitored using computer vision programs (from camera and laser sensor data) and engage with the driver or pilot using voice commands or prompts, rule out false positive conditions by asking more questions and direct safer maneuvering of the vehicle. The computer in the vehicle will use microphone to receive voice commands, send the data to central processing unit, then data will be sent back to computer vision program (data obtained from the camera and laser sensors) for validation and execute commands, if all turns out safe, the instruction of be sent to computer processing center as well voice response will be sent to speaker for the driver/pilot understanding. In addition, drivers can initiate video call or phone call using voice commands and set up destination using voice commands. Finally, Driving Assist can connect with all the IoT and convert any motor vehicle into a smart vehicle.

The Driving Assist computer (Robotic Assistant brain) can see using computer vision signals by the using signal input from camera and sensors (laser, radar, and ultrasonic) attached with it.

The Driving Assist computer (Robotic Assistant brain) can process GPS data, along with all signals from cameras, sensors, microphone and speaker and assist driving or help with auto-pilot driving.

The robot uses computer camera data and converts the received computer vision data into signals differentiate between human, animals, vehicle, distance of nearby objects, activity nearby vehicles and anticipate other vehicles moves based on pattern of their driving, assess risks based on surroundings and activate voice prompts to assist the driver.

Driving Assist can turn on or turn off vehicles using voice recognition technologies, where vehicle can speak and listen, by using voice commands when provided passwords or security information's.

Driving Assist speaks with humans when spotted in the range and asks for password and listen to response to disable alerts.

The Cognitive computation module (Robotic Assistant brain) listens using the Microphone attached with it.

The Driving Assist converts the received speech (of driver) to text data.

The converted text data (drivers' response) are submitted to the Driving Assist central processing unit (Robotic Algorithm) for analysis.

The Driving Assist utilizes cognitive computation algorithm which is artificial intelligence algorithm along with decision rules to analyze the human response, collects further information by listening or from cameras or sensors and delivering the specific assistance.

The Driving Assist collects targeted information from the customer/resident by sending text messages to Robot speakerphone which is converted to speech so that speakerphone can utilize the data signal.

The driver hears the speech from the Driving Assist speakerphone and responds to questions asked which Robotic Driving Assist can listen and process like human security or customer support assistant.

Driving assist connects with nearest vehicles and communicate road traffic information and risk of collision threats and alert the nearest vehicles drivers using voice commands to move out of the way form vehicle in front or behind to create space for vehicle in jeopardy.

Driving assist will be able to turn on auto pilot driving mode without the help of the human driver and safely drive the vehicle when requested or in a time of emergency using GPS, camera, laser sensor, radar sensor, ultrasonic sensor, blue tooth sensor, RFID tags and surrounding vehicle signals.

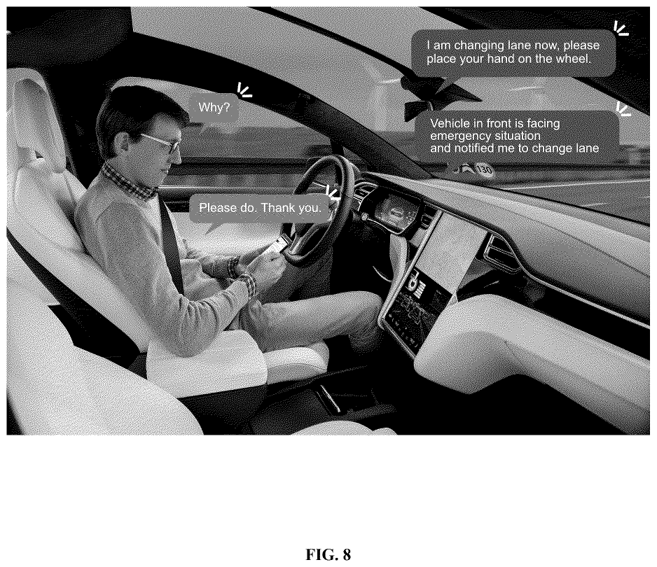

Referring now to FIG. 7, a block diagram of a computer implemented system is shown, in accordance with an exemplary embodiment of the present disclosure. The FIG. 8 to FIG. 11 explains how spoken conversation works between the driver and the vehicle. The figure used images of interior of the vehicle or the driver to demonstrate the speaking and listening functions of the artificial intelligence modules and or decision rules modules (the current invention does not claim credit for image of the interior of the car or the driver). The figure demonstrates that the vehicle can speak and listen to the driver. The driver listens to the voice spoken by the car or the vehicle while keeping an eye on the street and responds verbally to cars query like a normal human spoken conversation. This prevents drivers from taking away attention from the street and stay focused on driving while at the same time speaking and listening to the driving assist (or to the vehicle). Enabling the car or any vehicle's ability to speak (vehicle response or driving assist commands) and listen (driver's commands or drivers' response) adds significant addition to the current inventions which will reduce road traffic accidents and improve drivers' safety in the street.

Referring now to FIG. 8-11, a conversation between a driver/user and a computer implemented system of a vehicle to assist the driver/user while driving the vehicle is shown, in accordance with an exemplary embodiment of the present disclosure, wherein said conversation is spoken conversation (not text messages) through an in-built speaker and a microphone of said computer implemented system.

According to FIG. 8, the driver/user is present in the vehicle to drive the vehicle in auto pilot mode, i.e., the driver may be browsing internet on the phone and may expect assistance from the computer implemented system. During driving period, when the car determines an emergency situation and needs to change lane, said computer implemented system notifies the driver through a speaker coupled to said system that it is changing lanes according to one embodiment.

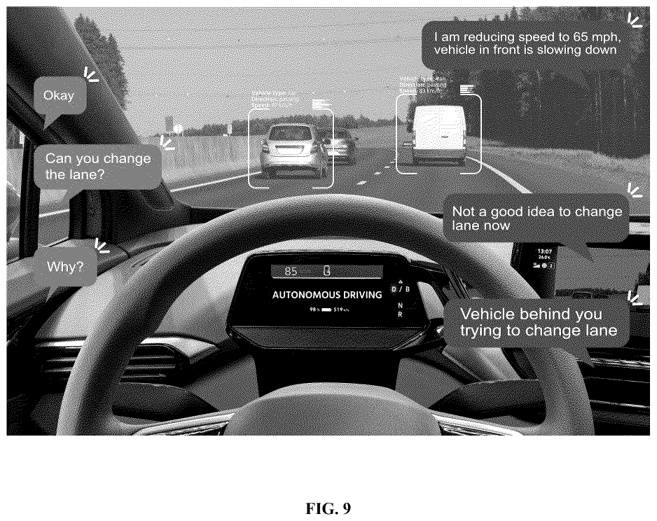

According to FIG. 9, when the vehicle is in autonomous driving mode (in auto pilot mode), the driving assist of the computer implemented system may be already turned on, and it performs spoken conversation with the user/driver according to one embodiment of the computer implemented system of vehicle. For example, if speed of another vehicle which is in front of the user's/driver's vehicle is slowing and due to which both the vehicles are closing in, said driving assist notifies the driver/user that it is going to reduce speed of the vehicle because the vehicle in front is slowing down. Further, the user/driver may ask whether it's possible to change the lane instead of slowing down, for which the driving assist first analyzes the situation, and if said assist determines that this is not good to change lane at that moment, it notifies the driver/user along with the reason of its denial.

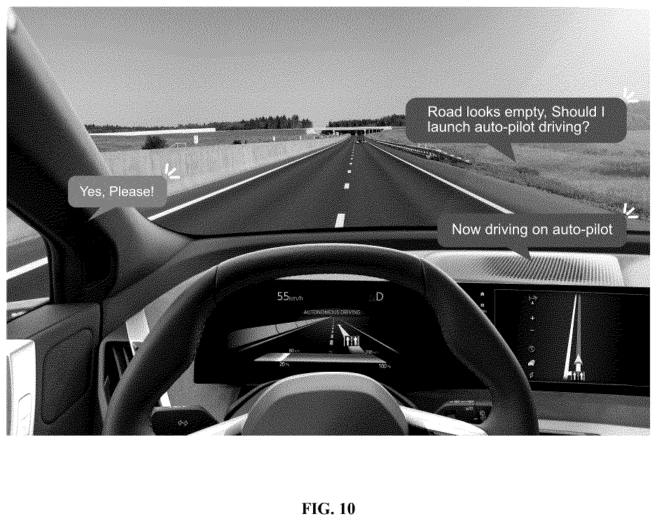

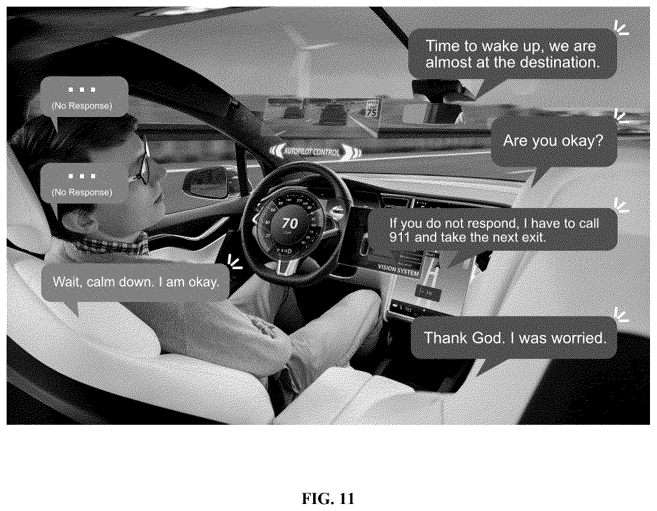

According to FIG. 10, when the vehicle is in autonomous driving mode, (in auto pilot mode), the driving assist may be already turned on according to one embodiment of the computer implemented system of vehicle, and it performs spoken conversation between the vehicle and the user/driver. According to one embodiment of the computer implemented system of vehicle, when the driving assist analyses that the road is empty or the number of vehicles is very less on the road, it suggests the driver to turn on auto-pilot driving mode, takes confirmation from the driver and consequently the auto-pilot driving mode is turned on. Furthermore, according to FIG. 11, the driver/user is present in the vehicle to drive the vehicle in auto pilot driving mode and expects assistance from the computer implemented system of the vehicle. During the auto-driving period, when the destination is about to come and the driver/user is asleep, the driving assist notifies the driver/user that he/she is almost at the destination according to one embodiment of the computer implemented system of vehicle. If the driver/user does not respond to this spoken notification, the driving assist asks about the health condition of the driver/user. In case the driver/user further fails to respond, the driving assist calls the emergency helpline number.

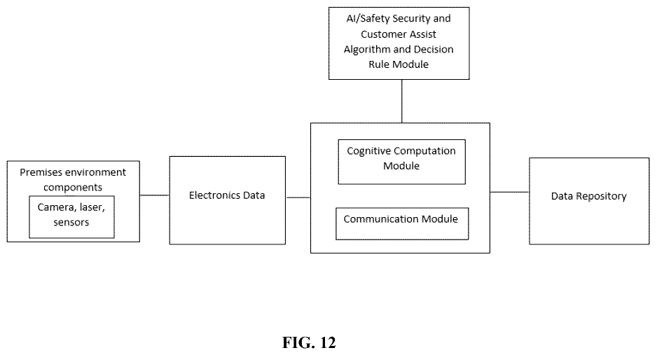

Referring now to FIG. 12, a block diagram of a computer implemented system provide security, safety and customer support in a premises is shown, in accordance with an exemplary embodiment of the present disclosure. The computer implemented system may include a cognitive computation module, a communication module, an Artificial intelligence module/Safety, Security and Customer Assist Algorithm with a decision rules module and a server. The cognitive computation module may be coupled to the communication module, the Artificial intelligence module/Safety, Security and Customer Assist Algorithm, the decision rules modules and the server. The cognitive computation module may be configured in a processor of the computing device. Further, the cognitive computation module of the computer implemented system to assist the human/user may be linked with electronic data which consists of data of premises surrounding. The cognitive computation module may access the electronic data as and when required to assist a human/user while providing security, safety and customer support in a premises. The electronic data may consist of data from the environment components like GPS, camera, speakerphone, microphone, and sensors as well as API connection with other IoT devices. The environment components may be responsible of capturing data outside the premises or premises surrounding. Furthermore, the cognitive computation module of the computer implemented system may be coupled to the other electronic records or data repository, network systems for fetching information of pattern of the possible security and safety related situations.

The Artificial intelligence module of the computer implemented system coupled with the cognitive computation module may contain all possible security and safety related situations, with all possible questions so that false positive situations are eliminated just by speaking and listening to answers to the questions in addition to using facial recognition data or camera input. The possible security and safety related situations may be intrusion, burglary, theft, fire, flood, or any natural disasters. The Artificial intelligence module may assist the cognitive computation module as and when required to provide security, safety and customer support in a premises.

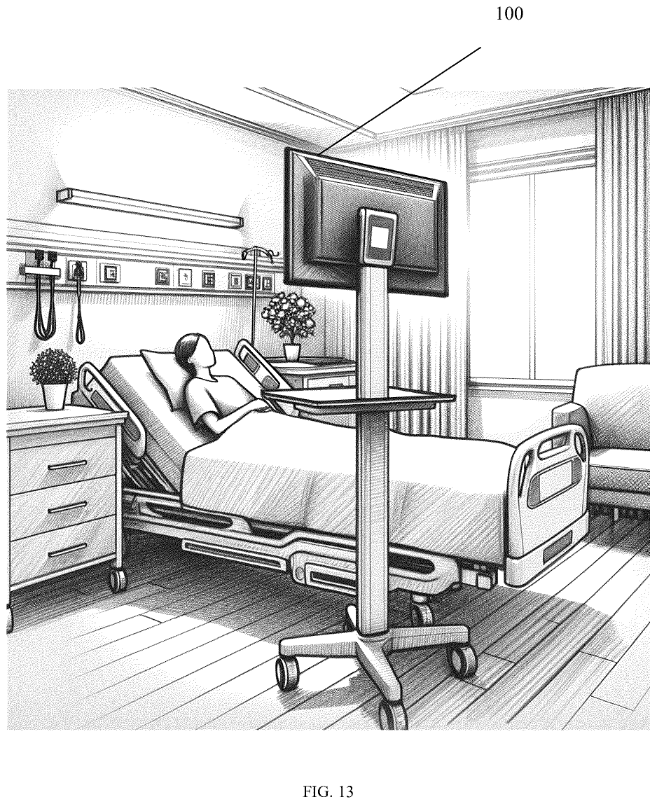

Referring now to FIG. 13, a block diagram of a computer implemented system (5000) is shown, in accordance with an exemplary embodiment of the present disclosure. The computer-implemented system (5000) for diagnosing diseases and providing treatments may include a first computing device (100) having a cognitive computation module (110), a second computing (200) device and a second communication module (120). The cognitive computation module (110) may be configured in a processor of a first computing device (100). The second computing device (200) may be coupled with a second communication module (120) which is an internet webpage. Further, the first computing device (100) and the second computing device (200) of the computer-implemented system may be interconnected via an internet connection. The cognitive computation module (110) may receive data inputs from the second computing device (200) and the received data may be analyzed by the cognitive computation module (110) of the first computing device (100) utilizing an artificial intelligence module and/or a decision rule module (130).

In one embodiment, the first computing device (100) and the second computing device (200) of the computer-implemented system (5000) may be one of a desktop, a mobile phone, a laptop, a tablet, a wearable device, a robot, etc. Further, the first computing device (100) may be having at least a speaker, a microphone, and/or a camera. The microphone may be used to receive the speech whenever the patient speaks. The speaker of the first computing device (100) to communicate with the patient like for example to ask questions. The camera of the first computing device (100) may be used to identify the physical position of or gesture of the patient. In one embodiment of the disclosure, the first computing device (100) speaks to the patient, listen to patient's response and communicate with a caregiver or provider via the speaker, the microphone, and/or the camera.

In one embodiment, the first computing device (100) and the second computing device (200) of the computer-implemented system (5000) may connect to one another from a remote location.

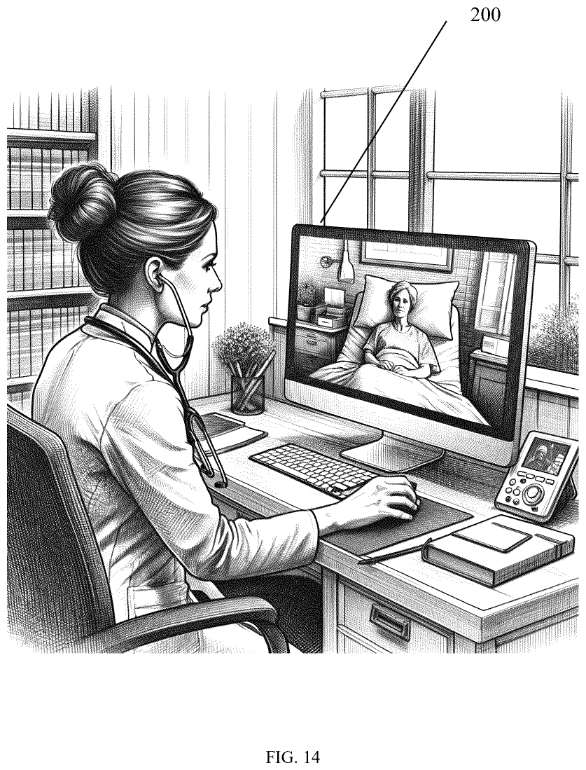

In one embodiment, the second computing device (200) of the computer-implemented system (5000) may be adapted to connect to the first computing device (100) from a remote location for a video or audio call. In the audio or video call, the first computing device (100) may be deployed in the vicinity of the one or more patient as shown in FIG. 4 and the second computing device (200) may be deployed in the vicinity of the doctor or any healthcare provider as shown in FIG. 14. The one or more patient may not have to operate the first computing device (100) to connect with the second computing device (200) which deployed in the vicinity of the doctor or any healthcare provider.

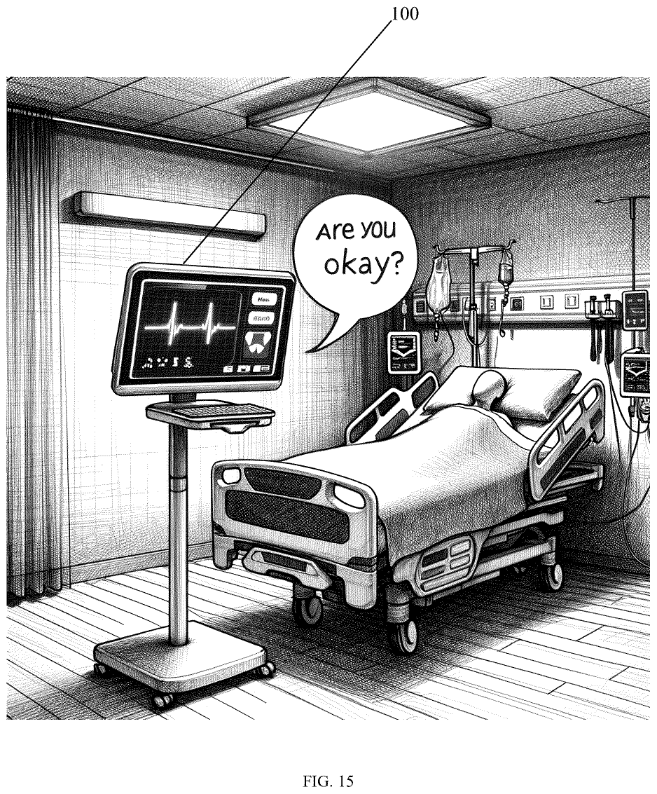

In one embodiment of the disclosure, either of the computing device (100), (200) may connect with one another as and when needed. For example, the second computing device (200) deployed in the vicinity of the doctor or any healthcare provider may connect to the first computing device (100) which deployed in the vicinity of the one or more patient for an audio or video call just by giving a voice command and vice versa. In one embodiment of the disclosure, the first computing device (100) deployed in the vicinity of the one or more patient may connect to the second computing device (200) deployed in the vicinity of the doctor or any healthcare provider by observing the physical gesture of the patient and/or by the voice command of the one or more patient. Further, the first computing device (100) deployed in the vicinity of the one or more patient may speak to the one or more patient when there is an abnormal finding in the patients' vital signs such as pulse oximetry data, blood pressure data, telemetry data, etc as shown in FIG. 15.

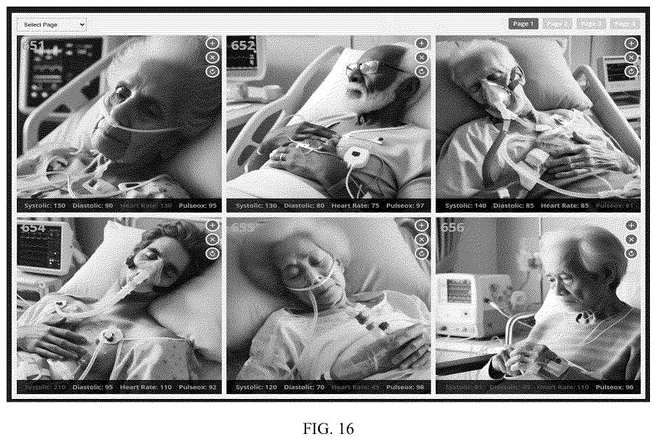

Referring now to FIG. 16, live video feed windows of one or more patients may be displayed on the second computing device (200) from one or more first computing devices (100), (200) of the computer-implemented system (5000). Each of the video feed window displays respective patient's telemetry data, and or pulse oximetry data and or vital signs data, and or any other data from electronic medical record, and or other remainders data to improve patient safety.

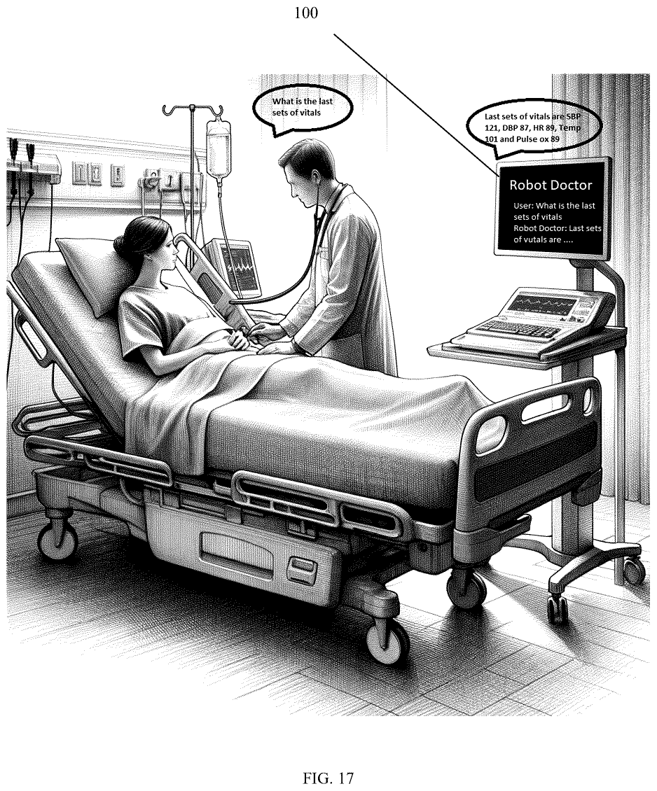

Referring now to FIG. 17, the first computing device (100) speaks/answers to the doctor's query regarding the patient's vitals, patient's health history, patient's medical reports data and other such patient's data. The first computing device may collect the patient's data from the electronic medical records.

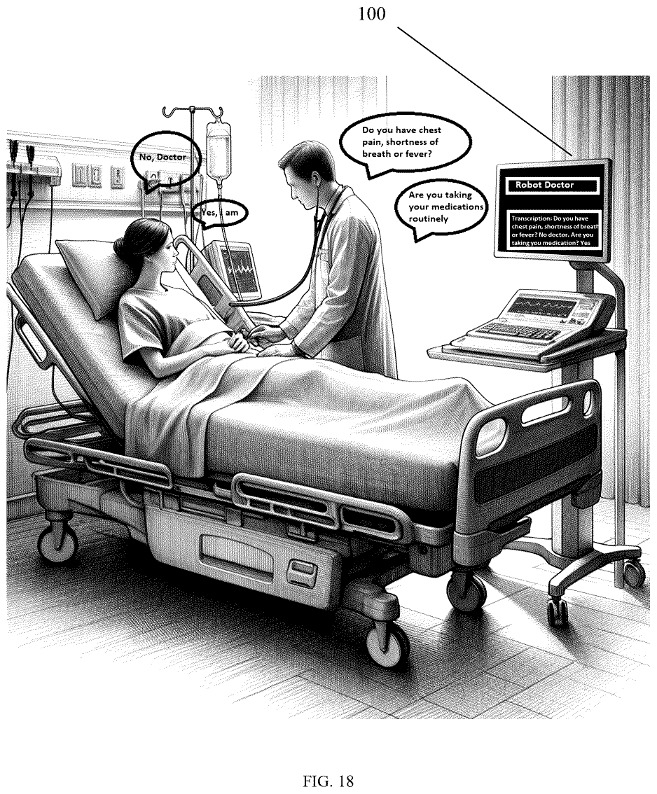

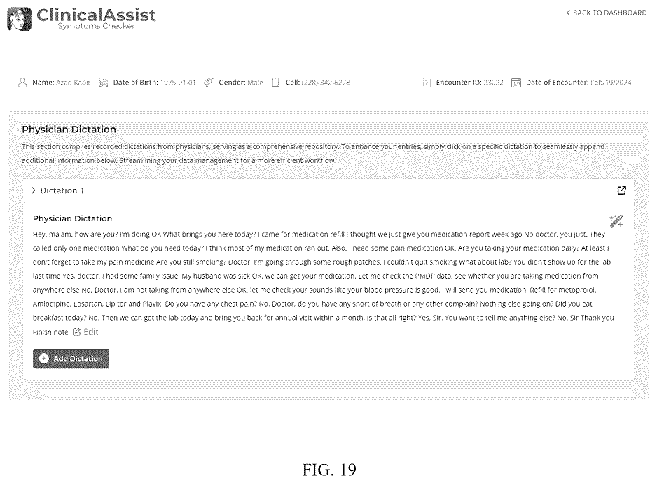

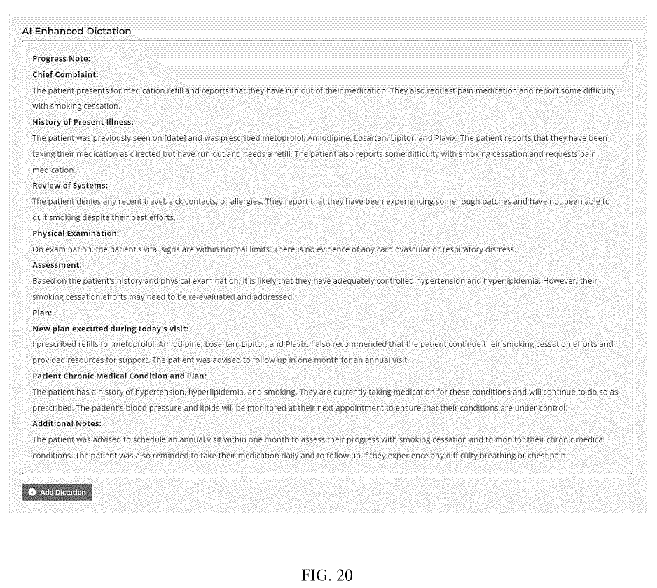

Referring now to FIG. 18, the first computing device (100) may transcribe the conversation between patient and healthcare providers. The transcription of the conversation may include at least one of the patient data such as but not limited to history of a patient, physician exam, laboratory data, radiological data, diagnosis data, and treatment data to create AI enhanced encounter notes. Furthermore, FIG. 19 shows an example of a patient doctor/physician conversation during the patient rounding and the first computing device transcribing them exactly as it is and subsequently transferring them to a website for doctor/physician view and modification using an edit button (to add modify and delete unnecessary component). Furthermore, FIG. 20 shows an example of creation of a doctor/physician encounter notes from the patient doctor/physician conversation during the patient rounding (which was transcribed by the first computing device) and converted to physician progress notes or encounter notes by using AI algorithm. It is designed to improve physician workflow and reduce work load.

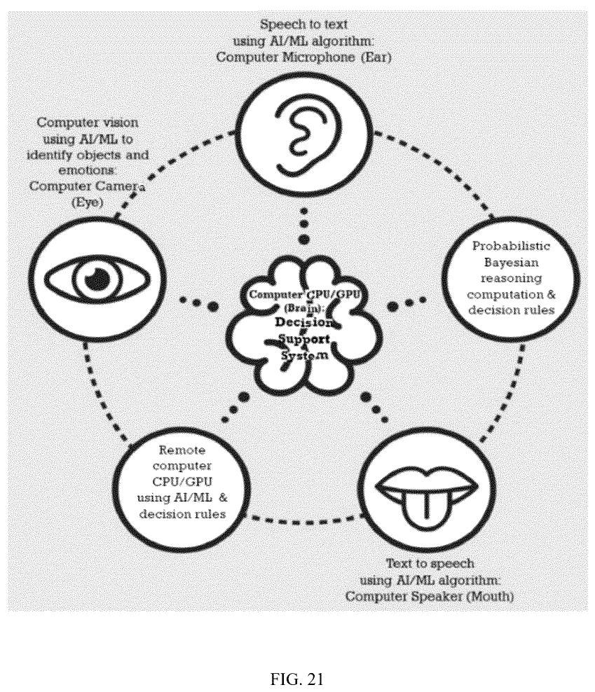

Referring now to FIG. 21, illustrates the operational pathways and elements of a cognitive computation system. It maps out the progression of data from initial sensory collection to advanced cognitive processing phases. This encompasses both the recursive feedback mechanisms and the algorithms for decision support, which synthesize the incoming data to generate an output. The system employs a microphone, speaker, camera, and a computing processing unit—either a CPU or GPU—to mimic human faculties: the ears, mouth, eyes, and brain, respectively, thereby achieving a human-like interactive capability.

The current invention refers to a cognitive computation system which is a computational system designed to mimic or simulate human cognitive functions, such as learning, reasoning, perception, and decision-making. The systems utilize some artificial intelligence (AI) and Machine learning (ML) algorithms and decision support algorithms to mimic human cognitive functions, where the purpose is to use AI/ML algorithms and decision support models to analyze complex data, learn from it, and make predictions or decisions similar to the way humans do.

Cognitive computation systems incorporate various techniques from machine learning, natural language processing, neural networks, and data analytics to process and analyze large amounts of data. They are designed to handle ambiguous and uncertain information, learn from new data inputs, and adapt over time. The current invention uses cognitive computation systems to assist in health care, provide driving assistance, protect homes and businesses from intrusion and burglary, provide safety and security, assist in identity verification, and many other functions. The invention aims to replicate the human brain's ability to deal with complex, ambiguous situations and to learn from experience. By doing so, it can support or augment human decision-making.”

The computing device of the computer implemented system to provide security, safety and customer support in a premises may be one of a desktop, a mobile phone, a laptop, a tablet, a wearable device, a robot, a personal digital assistant and the like. Further, the communication module of the computer implemented system to provide security, safety and customer support in a premises may include microphone, speaker, any sensor, a camera or a combination thereof. The communication module of the computer implemented system may be responsible for capturing data inside the premises. The microphone may be used to receive the speech whenever the driver/user speaks. The speaker may be used by the computer implemented system to communicate with the driver/user like for example to ask questions. The camera of the computer implemented system may be used to identify the physical position of or gesture of the driver/user. In one embodiment of the disclosure, the communication module may speak to the intruder and/or user/owner, listen to intruder's/user's response and communicate with an emergency number.

The cognitive computation module of the computer implemented system may serves as surrounding assistant by speaking out premises data, premises surrounding vehicle data and other data from the electronic records.

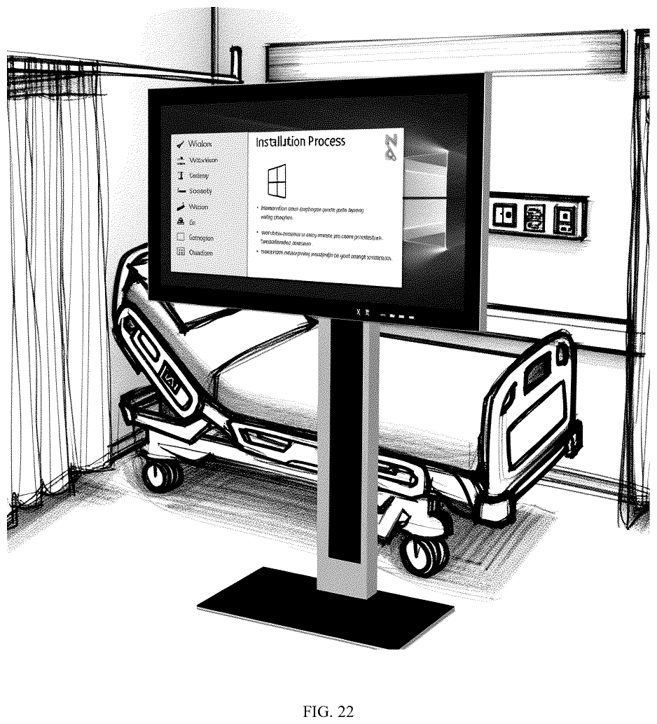

Referring to FIG. 22, the figure illustrates the installation of the cognitive computation module on a display. In accordance with one or more embodiments of the present invention, the cognitive computation module is provided as a .NET installer for Windows-based computers. It should be noted that the applicability of the cognitive computation module extends beyond Windows computers. The module is designed for compatibility across various computing platforms, including but not limited to Android, Apple (macOS and iOS), and Linux environments.

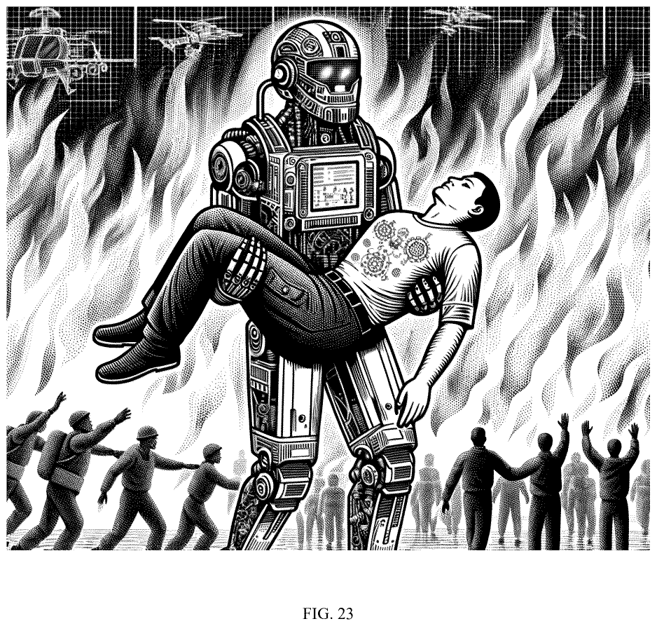

Referring to FIG. 23, the figure illustrates a humanoid robot rescuing a human from a hazardous wildfire situation. The integration of humanoid robots into wildfire rescue operations represents a groundbreaking advancement in enhancing safety and efficacy in combating forest fires and similar hazards. These robots, equipped with advanced sensors and AI, can navigate through intense fire zones, where the risks of smoke inhalation and direct flame exposure render human intervention highly perilous. Their design enables them to identify and rescue individuals trapped by fire, all the while relaying critical information back to emergency responders, including the location of survivors, fire intensity, and safe paths for evacuation. This not only significantly reduces the risk to human firefighters but also increases the chances of successful rescues in situations where every second counts. Moreover, the deployment of such robots could revolutionize firefighting strategies, allowing for more precise and targeted approaches to containment and extinguishment, ultimately contributing to more effective management and mitigation of wildfire incidents.

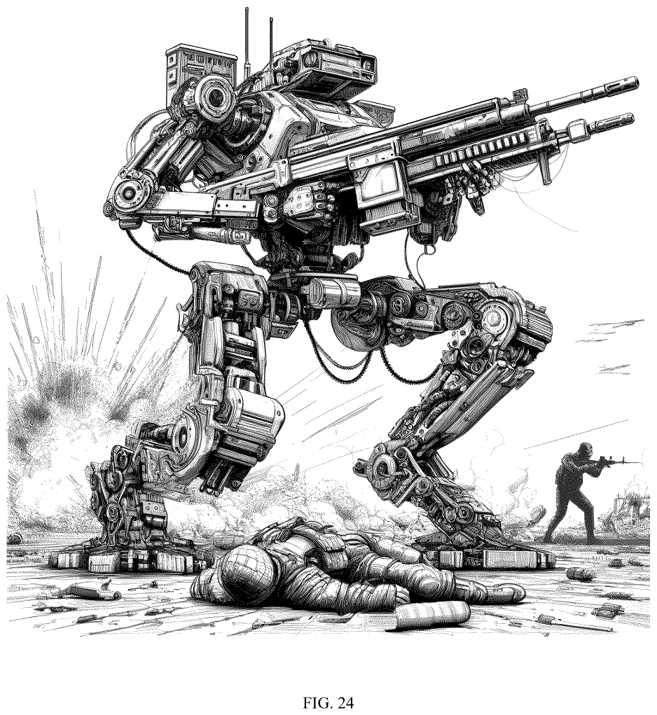

Referring to FIG. 24, the figure illustrates a humanoid robot rescuing a soldier from an active bullet fire situation on a battlefield. The deployment of humanoid robots in war zones for the purpose of rescuing humans represents a transformative approach to addressing the complexities of modern conflicts, gun violence, hostage situations, and other hazardous environments. These robots, designed to withstand the rigors of combat zones, can navigate through areas fraught with dangers such as active gunfire, explosive devices, and unpredictable threats, areas where the risk to human life is exceedingly high. Equipped with advanced navigation systems, real-time data processing capabilities, and robust physical frameworks, these robots can execute rescue missions with precision, swiftly locating and extracting civilians or soldiers from imminent danger. Their intervention minimizes the exposure of human rescue teams to life-threatening risks, ensuring that help can reach those in need without additional human casualties. Furthermore, the use of such robots can facilitate negotiations in hostage scenarios by safely delivering communications or aid without escalating tensions. Ultimately, the integration of humanoid robots into such high-stake environments can save lives, reduce injuries, and present new strategies in managing the complexities of modern warfare and conflict resolution.

The cognitive computation module may be configured in a processor of the computing device which can speak to, listen to and or see any intruder or/and user/owner. Further, the cognitive computation module may receive text data input from the communication module after converting speech to text input. The received text data may be analyzed by the cognitive computation module using the artificial intelligence and a decision rule module. The cognitive computation module creates a response of text output which may be converted to speech data using text to speech module and may send the speech data to the communication module to communicate the response. The cognitive computation module may receive further input from the intruder and/or owner/user of the premises using the communication module. The cognitive computation module may analyze all input and to assist the intruder and/or owner/user while driving a vehicle.

The cognitive computation module of the computer implemented system may serve as abnormal data monitoring system when such data become available in any electronic records. The cognitive computation module may speak to the owner/user, listen to owner's/user's response, and connect with an emergency number about to inform about premises or owner's/user's status.

The cognitive computation system used in patient care delivery, driving assist, safety, security, customer support, and identity validation system can communicate with the user using any possible language and also communicate with the monitoring system or other electronic system using their chosen language while keeping communication seamless and effective between users. The system works as a universal translator between users as two users can use two different languages while effectively communicating with each other.

The present disclosures should not be construed to be limited to the configuration of the method and system as described herein only. Various configurations of the systems are possible which shall also lie within the scope of the present disclosures.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the present disclosure.

The system as described in the disclosed teachings or any of its components may be embodied in the form of a computer system. Typical examples of a computer system include a general-purpose computer, a PDA, a cell phone, a programmed microprocessor, a micro-controller, a peripheral integrated circuit element, and other devices or arrangements of devices that are capable of implementing the steps that constitute the method of the disclosed teachings.

In a computer system comprising a general-purpose computer, such may include an input device, and a display unit. Specifically, the computer may comprise a microprocessor, where the microprocessor is connected to a communication bus. The computer may also include a memory the memory may include Random Access Memory (RAM) and Read Only Memory (ROM). The computer system further comprises a storage device it can be a hard disk drive or a removable storage drive such as a floppy disk drive, optical disk drive, and the like. The storage device can also comprise other, similar means for loading computer programs or other instructions into the computer system.

The computer system may comprise a communication device to communicate with a remote computer through a network. The communication device can be a wireless communication port, a data cable connecting the computer system with the network, and the like. The network can be a Local Area Network (LAN) or a Wide Area Network (WAN) such as the Internet and the like. The remote computer that is connected to the network can be a general-purpose computer, a server, a PDA, and the like. Further, the computer system can access information from the remote computer through the network.

The computer system executes a set of instructions that are stored in one or more storage elements in order to process input data. The storage elements may also hold data or other information as desired. The storage element may be in the form of an information source or a physical memory element present in the processing machine.

The set of instructions may include various commands that instruct the processing machine to perform specific tasks such as the steps that constitute the method of the disclosed teachings. The set of instructions may be in the form of a software program. The software may be in various forms such as system software or application software. Further, the software might be in the form of a collection of separate programs, a program module with a larger program or a portion of a program module. The software might also include modular programming in the form of object-oriented programming. The software program or programs may be provided as a computer program product, such as in the form of a computer readable medium with the program or programs including the set of instructions embodied therein. The processing of input data by the processing machine may be in response to user commands or in response to the results of previous processing or in response to a request made by another processing machine.

All the separate disclosures can be combined with an automated guided vehicle (AGV) or with an autonomous mobile robot (AMR) which follows along human or users or any premises as per guidance using radio waves, vision cameras, magnets, or lasers for navigation. All these computers implanted system can serve as a universal translator by adapting all language translation for users' convenience. In addition, all the separate disclosures can be combined together to provide combined disclosures to provide healthcare, safety, security, and or customer support at home or institutes that is powered by humanoid robots.

This invention is a Cognitive Computation Framework, not an AI. It is a computation of human mind. Building a cognitive computation model is a multifaceted endeavor that integrates a diverse array of algorithms and techniques, both from artificial intelligence (AI) and other fields. At its core, such a model aims to replicate human-like cognitive processes (human mind), including perception, reasoning, learning, and decision-making. To achieve this, machine learning algorithms such as supervised learning, unsupervised learning, and reinforcement learning are employed to discern patterns and associations from data, while deep learning models like convolutional neural networks (CNNs) and recurrent neural networks (RNNs) handle tasks involving image processing and sequential data analysis. Additionally, non-AI algorithms such as probabilistic reasoning, Bayesian model, logic, and search algorithms play crucial roles in knowledge representation, problem-solving, and decision-making. There is no other known Cognitive computation framework existing in the market.

The key aspects that set us apart from other artificial intelligence programs is our ability to reason, understand complex concepts, and establish meaningful connections between words. Unlike popular generative AI systems such as ChatGPT, Alexa, Google, or Siri, Cognitive computation goes beyond superficial interactions to predict the next best word and utilizes a cognitive computation algorithm that closely mimics the human thinking process. It is essential to note that ChatGPT, Alexa, Google, or Siri, while impressive, cannot independently improve healthcare providers' workflows, diagnose patients, or provide treatment solely based on a patient encounter through a patient-facing application. Consequently, it is important to understand that Cognitive computation should not be considered a comparison to any other existing generative AI or AI systems available in the market.

While these existing AI platforms provide valuable services, they often fall short when it comes to comprehensive understanding and reasoning. Clinical Assist, on the other hand, excels in bridging the gap between human cognition and AI capabilities specially how doctors think. Our advanced algorithm allows us to analyze medical data, interpret patient needs, and provide insightful recommendations that are aligned with the context and complexity of real-world healthcare scenarios which is aligned with provider's workflow.

Below is a comparison table between cognitive computation module (or frameworks), and Generative AI:

| | Cognitive | |
|---|---|---|
| Aspect | Computation Module | Generative AI |
| Objective | Simulate human-like | To generate new |
| | cognitive processes | content or data |
| | (human mind) | |
| Focus | Understanding and | Creating new content |
| | mimicking human | based on given |
| | cognition (mind | parameters |
| Key Techniques | Knowledge | Neural networks, |
| | representation, reasoning, | deep learning, |
| | learning | probabilistic models |
| Applications | Robotics | Creative content |
| | | generation, art, music, |
| | | text generation |
| Data | Often relies on structured | May require large |
| Requirements | data and ontologies | datasets for training |
| Output | Focuses on logical | Output is typically |
| Interpretation | reasoning and | creative and may lack |
| | decision-making | interpretability |
| Human | May involve human- | Less emphasis on |
| Interaction | like interfaces and | direct interaction with |
| | conversational agents | users |
| Complexity | Can be complex due | Complexity depends |
| | to modeling human | on the domain and |
| | cognitive processes | the desired output |
| Learning | May incorporate supervised, | Primarily utilizes |
| Paradigm | unsupervised, and | unsupervised learning |
| | reinforcement learning | and reinforcement learning |
| Use Cases | Decision support systems | Image generation, text |
| | | generation, style transfer |
| Performance | May include cognitive | Quality of generated |
| Metrics | plausibility, accuracy | content, diversity, |
| | in reasoning tasks | and coherence |

What is claimed is:

1. A computer implemented system to diagnose a disease and or provide treatments comprising:
a cognitive computation module configured in a processor of the computing device which can perform at least one task of speaking to, listening to and or seeing any patient or any user;
wherein the cognitive computation module receives text data input from a user using coupled communication module after converting speech to text input;
wherein received text data analysed by using artificial intelligence modules and or decision rules modules;
wherein the cognitive computation module creates a response of text output which is converted to speech data using text to speech module and then send to the communication module to communicate with the user.

2. The computer implemented system to diagnose a disease and or provide treatment of claim 1, wherein the cognitive computation module may receive further speech input from the user using the communication modules.

3. The computer implemented system to diagnose a disease and or provide treatment of claim 1, wherein the cognitive computation module transcribes conversation between patient and healthcare providers which includes at least one of the patient data such as but not limited to history of a patient, physician exam, laboratory data, radiological data, diagnosis data, and treatment data to create AI enhanced encounter note.

4. A computer implemented system to diagnose a disease and or provide treatments comprising:
a cognitive computation module configured in a processor of the computing device which can perform at least one task of speaking to, listening to and or seeing any patient or any user;
wherein the cognitive computation module receives text data input from a user using coupled communication module after converting speech to text input;
wherein received text data analysed by using artificial intelligence modules and or decision rules modules;
wherein the cognitive computation module creates a response of text output which is converted to speech data using text to speech module and then send to the communication module to communicate with the user;
wherein the cognitive computation module may receive further speech input from the user using the communication module,
wherein the cognitive computation module documents history of patient, and or diagnosis and or treatment data in to an encounter note for the patient.

5. The computer implemented system to diagnose a disease and or provide treatment of claim 1, wherein the cognitive computation module is coupled/linked with all available data in the electronic medical records which consists at least one patient data about patient's signs, symptoms, differential diagnosis, past medical history, past surgical history, social history, medications, allergies, laboratory findings, radiological findings, physician's notes, nurse's notes, and treatment data.

6. The computer implemented system to diagnose a disease and or provide treatment of claim 1, wherein the computing device is one of a desktop, a mobile phone, a laptop, a tablet, a wearable device, a robot, etc.

7. The computer implemented system to diagnose a disease and or provide treatment of claim 1, wherein the communication module consists of either a microphone, speaker, any sensor, RFID sensor, a camera, Bluetooth devices, Wi-Fi devices, Cellular devices, blood pressure cuff, pulse oximetry devices, telemetry devices, blood sugar meter, weighing scale or a combination thereof.

8. The computer implemented system to diagnose a disease and or provide treatment of claim 1, wherein the camera identifies the physical position of or gesture of the patient; and the communication module speak to the patient, listen to patient's response and communicate with a caregiver or provider.

9. The computer implemented system to diagnose a disease and/or provide treatment of claim 1, wherein the cognitive computation module can help connect to a video call without needing any touch or click from the patient facing device or the user device;
wherein the video call is initiated from a remote location using either a laptop, desktop, tablet, robot, a wearable device, any computing device or a mobile phone.

10. The computer implemented system to diagnose a disease and/or provide treatment of claim 1, wherein the cognitive computation module transmits live stream of the computer video to a remote location which can be viewed either from a laptop, desktop, tablet, robot, a wearable device, any computing device or a mobile phone.

11. The computer implemented system to diagnose a disease and or provide treatment of claim 1, wherein the cognitive computation module is connected to at least one record of electronic medical records or data repository, network systems for fetching information, medical data, etc.

12. The computer implemented system to diagnose a disease and/or provide treatment of claim 1, wherein the cognitive computation module serves as providers rounding assistant by speaking out at least one patient information of patient's vitals, laboratory data, radiological data, microbiology data, providers notes and other data from the electronic medical records.

13. The computer implemented system to diagnose a disease and or provide treatment of claim 1, wherein the cognitive computation module serves as abnormal data monitoring system when such data become available in any electronic medical records, wherein the cognitive computation module performs at least one task of speaking to the patient, listen to patient's response, and or communicate with a caregiver or provider about patient clinical status.

14. A method for diagnosing disease and or providing treatment, comprising:
speaking with patient about patient's symptoms;
listening to patients' response and asking further questions based on patients' response; and/or
transmitting patient's symptoms data to a cognitive computation module configured in a processor of a computing device;
analysing all the symptoms data using the cognitive computation module to diagnose a disease or a medical condition;
providing suitable treatment recommendation for the diagnosed disease or medical condition.

15. The method for diagnosing disease and or providing treatment of claim 14, wherein the cognitive computation module documents at least one information of patient history, and or diagnosis and or treatment data into an encounter note for the patient.

16. The method for diagnosing disease and or providing treatment of claim 14, wherein the computing device is one of a desktop, a mobile phone, a laptop, a tablet, a wearable device, a robot, etc.

17. The method for diagnosing disease and or providing treatment of claim 14, wherein the communication with the patient and providing suitable treatment done via a communication module, wherein the communication module is either a microphone, speaker, sensor, RFID tags, Bluetooth devices, Wi-Fi devices, Cellular devices, a camera or a combination thereof.

18. The method for diagnosing disease and or providing treatment of claim 14, wherein the speaking and listening to the patient complains comprises questions, wherein every subsequent question is based on the analysis of the first/previous question asked by the cognitive computation module.

19. The method for diagnosing disease and or providing treatment of claim 14, wherein the cognitive computation module is adapted to monitor the patient and generate instructions for the patient or to a caregiver based on physical gesture or movements of the patient.

20. The method for diagnosing disease and/or providing treatment of claim 14, wherein the cognitive computation module is linked with at least one information of available signs, symptoms, laboratory findings, radiological findings and treatment data in the electronic medical records.

21. The method for diagnosing disease and or providing treatment of claim 14, wherein the server is adapted to connect to at least one system of other servers, networks, systems for fetching information, medical data, etc.

22. The computer implemented system to diagnose a disease and or provide treatment of claim 1, wherein the cognitive computational module is adapted to perform at least one task of shutting down the computer device, placing the device in sleep mode, or increasing or decreasing speaker volume or searching any website for requested information when receives voice command.

23. The computer implemented system to diagnose a disease and or provide treatment of claim 1, wherein the cognitive computational module is adapted to receive input from at least one of camera and or microphone and determine patient severity of illness.

24. The computer implemented system to diagnose a disease and or provide treatment of claim 1, wherein the cognitive computational module is adapted to write a provider progress note or encounter note which includes at least one of patient chief complaints, history of present illness, review of system and treatments.

25. The computer implemented system to diagnose a disease and/or provide treatment of claim 1, wherein the cognitive computational module is adapted to perform at least one task of speaking and or listening to user response to deliver either cost-effective treatment strategies, and or reduce unnecessary testing, and or clinical documentation improvement messages and or any other messages.

26. The computer implemented system to diagnose a disease and or provide treatment of claim 1, wherein the cognitive computational module is adapted to perform at least one task of speaking and or listening to patients and educate patients about almost all common medical symptoms and diagnoses.

27. The computer implemented system to solve diagnose a disease and/or provide treatment of claim 1, wherein the cognitive computational module is adapted to perform at least one task of speaking and or listening in any language and submit the response or transcript of conversation or encounter note in English or any chosen language.

28. The computer implemented system to solve diagnose a disease and/or provide treatment of claim 1, wherein the cognitive computational module is adapted to send alert for diagnosis of sepsis or severe sepsis or septic shock.

29. The computer implemented system to solve diagnose a disease and/or provide treatment of claim 1, wherein the cognitive computational module is adapted to send alert for any diagnosis or life-threatening diagnosis.

30. The computer implemented system to solve diagnose a disease and/or provide treatment of claim 1, wherein the cognitive computational module is adapted to send alert for life threatening patient condition when abnormal or critical data is available.

31. The method for diagnosing disease and or providing treatment of claim 14, wherein the cognitive computational module is adapted to send alert for diagnosis of sepsis or severe sepsis or septic shock.

32. The method for diagnosing disease and or providing treatment of claim 1, wherein the cognitive computational module is adapted to transmit patient's vital signs collected from connected devices to a remote location for monitoring of the patient.

33. The method for diagnosing disease and or providing treatment of claim 14, wherein the cognitive computational module is adapted to send alert for any diagnosis or life-threatening diagnosis.

34. The computer implemented system to solve diagnose a disease and/or provide treatment of claim 1, wherein the cognitive computational module is adapted to provide at least one recommendation about patient visiting emergency room or contacting physicians or nurses for further healthcare.

35. The method for diagnosing disease and or providing treatment of claim 14, wherein the cognitive computational module is adapted to provide recommendation about visiting emergency room or contacting a physician or a nurse for further healthcare.

36. The computer implemented system to solve diagnose a disease and/or provide treatment of claim 1, wherein the cognitive computational module is adapted to notify users about conditions of patients waiting to be seen.

37. The method for diagnosing disease and or providing treatment of claim 14, wherein the cognitive computational module is adapted to notify users about conditions of patients waiting to be seen.

38. A computer implemented system to assist a human or user while driving a vehicle comprising:
a cognitive computation module configured in a processor of the computing device which can perform at least one task of speaking to, listening to and/or seeing driver/user or surroundings of the vehicle or streets;
wherein the cognitive computation module receives text data input from a coupled communication module after converting speech to text input;
wherein received text data analysed by the cognitive computation module using a cognitive computation module which includes artificial intelligence modules and/or decision rules modules;
wherein the cognitive computation module creates a response of text output which is converted to speech data using text to speech module and then send to the communication module to speak the response;
wherein the cognitive computation module may receive further speech input from the driver/user using the communication module;
wherein the cognitive computation module analyses all input and execute the best option to assist the driver/user in the vehicle.

39. A computer implemented system to assist a human or user while driving a vehicle comprising:
a cognitive computation module configured in a processor of the computing device which can perform at least one task of speaking to, listening to and or seeing driver/user or surroundings of the vehicle or streets;
wherein the cognitive computation module receives text data input from a coupled communication module after converting speech to text input;
wherein received text data analysed by the cognitive computation module using a cognitive computation module which includes artificial intelligence modules and or decision rules modules;
wherein the cognitive computation module creates a response of text output which is converted to speech data using text to speech module and then send to the communication module to speak the response.

40. The computer implemented system to assist human/user while driving a vehicle of claim 39, wherein the cognitive computation module may receive further speech input from the driver/user using the communication module.

41. The computer implemented system to assist human/user while driving a vehicle of claim 40, wherein the cognitive computation module analyses all input and execute the best option to assist the driver/user in the vehicle.

42. The computer implemented system to assist human or user while driving a vehicle of claim 38, wherein the cognitive computation module is coupled with at least one electronics data from either GPS, camera, laser sensor, radar sensor, ultrasonic sensor, Bluetooth devices, RFID tags, Wi-Fi connected devices, Cellular connected devices, and or surrounding vehicle signals or combination thereof.

43. The computer implemented system to assist human or user while driving a vehicle of claim 38, wherein the Artificial intelligence module/driving assist algorithm is coupled with the cognitive computation module with all possible road security and safety related rules and regulation data.

44. The computer implemented system to assist human or user while driving a vehicle of claim 38, wherein the computing device is at least one of a mobile phone, a laptop, a desktop, a tablet, a wearable device, a robot, personal digital assistant fitted in the vehicle, etc.

45. The computer implemented system to assist human or user while driving a vehicle of claim 38, wherein the communication module consists of either a microphone, speaker, any sensor, RFID tags, Bluetooth devices, a camera, Wi-Fi devices, Cellular devices, or a combination thereof.

46. The computer implemented system to assist human or user while driving a vehicle of claim 38, wherein the camera identifies at least one user or driver physical position, movements or gesture; and the communication module performs at least one task like speak to the driver/user, listen to driver's/user's response and or connect with an emergency number.

47. The computer implemented system to assist human or user while driving a vehicle of claim 39, wherein the cognitive computation module is connected to at least one of a data repository, and or network systems for fetching information of traffic rules, etc.

48. The computer implemented system to assist human or user while driving a vehicle of claim 39, wherein the cognitive computation module serves as surrounding assistant by speaking out at least one information about vehicle data, surrounding vehicle data, road condition, traffic condition and other data from the electronic records.

49. The computer implemented system to assist human or user while driving a vehicle of claim 39, wherein the cognitive computation module serves as abnormal data monitoring system when such data become available in any vehicle electronic records;
wherein the cognitive computation module performs at least one task of speaking to the driver or user, listening to driver's or user's response, and connecting with an emergency number about to inform driver's/user's status.

50. The computer implemented system to assist human/user while driving a vehicle of claim 39, wherein the cognitive computation module determines if the user or driver is medically incapacitated, and then activates auto pilot driving mode.

51. The computer implemented system to assist human or user while driving a vehicle of claim 39, wherein the cognitive computation module determines if street is comparatively empty or have fewer traffic and speaks to the user/driver to activate auto pilot driving mode.

52. The computer implemented system to assist human or user while driving a vehicle of claim 39, wherein the cognitive computation module speaks to the driver to change lane or take an exit or come to a complete stop.

53. The computer implemented system to assist human or user while driving a vehicle of claim 39, wherein the cognitive computation module speaks to the driver before taking over the driving as auto pilot mode.

54. The computer implemented system to assist human or user while driving a vehicle of claim 39, wherein the cognitive computation module speaks to the driver to place hands on the steering wheel.

55. The computer implemented system to assist human or user while driving a vehicle of claim 39, wherein the cognitive computation module speaks to the driver to verify password upon user/drivers' entry into the vehicle;
wherein the cognitive computation module validates the password if correct then turn on ignition.

56. The computer implemented system to assist human or user while driving a vehicle of claim 39, wherein the driver/user voice commands to perform at least one task of driving to a specific destination, turning on ignition, increasing or decreasing volume, increasing or decreasing temperature, initiating a phone call, initiating a video call, searching nearest restaurant, searching any location, managing the personal calendar, reading recent emails, messaging, or posting in social media using voice commands.

57. The computer implemented system to assist human or user while driving a vehicle of claim 40, wherein the driver/user voice commands to perform at least one task of driving to a specific destination, turning on ignition, increasing or decreasing volume, increasing or decreasing temperature, initiating a phone call, initiating a video call, searching nearest restaurant, searching any location, managing the personal calendar, reading recent emails, messaging, or posting in social media.

58. The computer implemented system to assist human or user while driving a vehicle of claim 39, wherein the cognitive computation module asks any human who is trying to open the car door or come close to the vehicle to provide entry password;
wherein the cognitive computation module validates the password and if correct then allows the human to enter inside the vehicle.

59. The computer implemented system to assist human or user while driving a vehicle of claim 58, wherein the cognitive computation module report to the emergency contact number if incorrect password is provided.

60. A computer implemented system to provide security, safety or customer support in a premises comprising:
a cognitive computation module configured in a processor of the computing device which can perform at least one task of speaking to, listening to and/or seeing a user;
wherein the cognitive computation module receives text data input from a coupled communication module after converting speech to text input;
wherein received text data analysed by the cognitive computation module using an artificial intelligence modules and decision rules modules;
wherein the cognitive computation module creates a response of text output which is converted to speech data using text to speech module and then send to the communication module to speak to the response;
wherein the cognitive computation module receives further input from the user using the communication module;
wherein the cognitive computation module analyses all input and provide security, safety or customer support in a premises.

61. The computer implemented system to provide security, safety or customer support in a premises of claim 60, wherein the cognitive computation module is coupled/linked with at least one of the premises environment components like GPS, camera, speakerphone, microphone, Bluetooth devices, Wi-Fi devices, Cellular connected devices, or other sensors, RFID tags, ultrasonic sensors, or API connection with other IoT devices to access data of the premises.

62. The computer implemented system to provide security, safety or customer support in a premises of claim 60, wherein the Artificial intelligence module/Safety, Security and Customer Assist module is coupled with the cognitive computation module with all possible security and safety related situations, with all possible questions so that false positive situations are eliminated just by speaking and listening to answers to the questions in addition to using facial recognition data or camera input.

63. The computer implemented system to provide security, safety or customer support in a premises of claim 60, wherein one of the possible security and safety related situations is intrusion, burglary, theft, fire, flood, or any natural disasters.

64. The computer implemented system to provide security, safety or customer support in a premises of claim 60, wherein the computing device is in one of a desktop computer, mobile phone, a laptop, a tablet, a wearable device, a robot, a personal digital assistant, etc.

65. The computer implemented system to provide security, safety or customer support in a premises of claim 41, wherein the communication module consists of either microphone, speaker, Bluetooth devices, Cellular connected devices, Wi-Fi devices, RFID tags, ultrasonic sensors, any sensor, a camera or a combination thereof.

66. The computer implemented system to provide security, safety or customer support in a premises of claim 60, wherein the camera identifies at least one of intruder, burglary, theft, fire, flood, or any natural disasters and the communication module which recognize human presence using camera, then verify by speaking to the user or intruder, listening to user's or intruder's response and or connecting with an emergency number.

67. The computer implemented system to provide security, safety or customer support in a premises of claim 60, wherein the cognitive computation module is connected to at least one of a data repository, network systems for fetching information of pattern of intrusion, burglary, theft, fire, flood, or any natural disasters, etc.

68. The computer implemented system to provide security, safety or customer support in a premises of claim 60, wherein the cognitive computation module serves as surrounding assistant by speaking out at least one of a premises data, premises surrounding and or other data from the electronic records.

69. The computer implemented system to provide security, safety or customer support in a premises of claim 60, wherein the cognitive computation module serves as abnormal data monitoring system when such data become available in any electronic records, wherein the cognitive computation module performs at least one task of speaking to the user, listening to user's response, and/or connecting with an emergency number about to inform premises or owner's/user's status.

70. A computer implemented system to validate identity of a user or a human comprising:
a cognitive computation module implemented in a processor of the computing device which can perform at least one task of speaking to, listening to and/or seeing a user;
wherein a cognitive computation module identifies the face of a human or a user using computer connected camera;
wherein the cognitive computation module uses text to speech module which is sent to the communication module to asks the human or user to provide the password; wherein the cognitive computation module receives response from human or user which is converted to text data input from a coupled communication module after converting speech to text input;
wherein received text data analysed by the cognitive computation module using an artificial intelligence module and or decision rule modules;
wherein the cognitive computation module analyses all input and provide identity validation for any user.

71. A computer implemented system to validate identity of a user or a human comprising of claim 70, wherein the cognitive computation module is coupled with Artificial intelligence modules and or decision rules modules which contains all possible organizational safety, security rules and or regulation data.

72. A computer implemented system to validate identity of a user or a human comprising of claim 70, wherein the computing device is in one of a mobile phone, a laptop, a desktop, a tablet, a wearable device, a robot, personal digital assistant in any commercial or residential premises.

73. A computer implemented system to validate identity of a user or a human comprising of claim 70, wherein the communication module consists of either microphone, speaker, any sensor, RFID tags, Bluetooth devices, a camera, Wi-Fi devices, Cellular connected devices, or a combination thereof.

74. A computer implemented system to validate identity of a user or a human comprising of claim 70, wherein at least one of a RFID tag or a Bluetooth device or Wi-Fi devices or cellular connected device or a key fob or any other sensors, is used to identifies presence of a person with or without using the computer camera data.

75. A computer implemented system to validate identity of a user or a human comprising of claim 70, wherein the user can sign in any computer using voice commands.

76. A computer implemented system to validate identity of a user or a human comprising of claim 70, wherein a user can sign in any computer and search internet for any information using voice commands.

77. A computer implemented system to validate identity of a user or a human comprising of claim 70, wherein the user can perform at least one of the tasks of bank transaction, access social media, post in social media, access any email or any protected accounts, unlock any device, unlock any computer, turn on any device or machinery, lock or unlock house or commercial facilities, lock or unlock a vehicle, turn on or turn off electronic machinery using voice commands.

78. A computer-implemented system for diagnosing diseases and providing treatments, comprising:
a cognitive computation module configured in a processor of a first computing device, equipped with at least a speaker, a microphone, and/or a camera;
a second computing device, coupled with a communication module which is an internet webpage;
the cognitive computation module of the first computing device receives data input from the second computing device;
the received data is analyzed by the cognitive computation module of the first computing device utilizing an artificial intelligence module and/or a decision rule module.

79. The computer-implemented system for diagnosing diseases and/or providing treatments as claimed in claim 78, wherein the first computing device and the second computing device are interconnected via an internet connection.

80. The computer implemented system to diagnose a disease and/or provide treatment of claim 78, wherein the first computing device and the second computing device is one of a desktop, a mobile phone, a laptop, a tablet, a wearable device, a robot, etc.

81. The computer implemented system to diagnose a disease and/or provide treatment of claim 78, wherein the first computing device and the second computing device connects to one another from a remote location.

82. The computer implemented system to diagnose a disease and/or provide treatment of claim 78, wherein the second computing device is adapted to connect to the first computing device from a remote location for a video or audio call.

83. The computer-implemented system to diagnosing a disease and/or provide treatment as claim 78, wherein live video feed window of one or more patients can be displayed on the second computing device from one or more first computing devices.

84. The computer implemented system to diagnose a disease and/or provide treatments of claim 83, wherein one or more patient's live video feed window displays respective patient's telemetry data, and or pulse oximetry data and/or vital signs data, and/or any other data from the electronic medical record, and or/other remainders data to improve patient safety.

85. The computer implemented system to diagnose a disease and/or provide treatments of claim 78, wherein the conversation between patient and doctor/healthcare providers is transcribed which includes at least one of the patient data such as but not limited to history of a patient, physician exam, laboratory data, radiological data, diagnosis data, and treatment data to create AI enhanced encounter notes.

86. A computer-implemented system for diagnosing diseases and providing treatments, comprising:
a cognitive computation module configured within a processor of a computer server coupled with internet connection;
a second computing device, coupled with a communication module which is an internet webpage, equipped with at least a speaker, a microphone, and/or a camera;
a third computing device, coupled with a communication module which is an internet webpage, equipped with at least a speaker, a microphone, and/or a camera;
the cognitive computation module, coupled within a processor of a computer server, receives data input from the second computing device or the third computing device;
the received data is analyzed by the cognitive computation module of the computer server utilizing an artificial intelligence module and/or a decision rule module.

87. The computer-implemented system for diagnosing diseases and/or providing treatments as claimed in claim 86, wherein the second computing device and the third computing device are interconnected via an internet connection.

88. The computer implemented system to diagnose a disease and/or provide treatment of claim 86, wherein the second computing device and the third computing device is one of a desktop, a mobile phone, a laptop, a tablet, a wearable device, a robot, etc.

89. The computer implemented system to diagnose a disease and/or provide treatment of claim 86, wherein the second computing device and the third computing device connects to one another from a remote location.

90. The computer implemented system to diagnose a disease and/or provide treatment of claim 86, wherein the second computing device is adapted to connect to the third computing device from a remote location for a video or audio call.

91. The computer implemented system to diagnose a disease and/or provide treatments of claim 86, wherein the conversation between patient and doctor/healthcare providers is transcribed which includes at least one of the patient data such as but not limited to history of a patient, physician exam, laboratory data, radiological data, diagnosis data, and treatment data to create AI enhanced encounter notes.

92. A system to create a humanoid robot comprising:
a cognitive computation module configured in a processor of the computing device which can perform at least one task of speaking to, listening to and/or seeing a user;
wherein the cognitive computation module receives text data input from a coupled communication module after converting speech to text input;
wherein cognitive computation module receives text data input from a coupled camera after analysing data using computer vision program;
wherein received text data analysed by the cognitive computation module using an artificial intelligence modules and decision rules modules;
wherein the cognitive computation module creates a response of text output which is converted to speech data using text to speech module and then send to the communication module to speak to the response;
wherein the cognitive computation module receives further input from the user using the communication module;
wherein the cognitive computation module analyses all input and work like a humanoid robot.

93. The system to create a humanoid robot comprising of claim 92, further comprising:
an interface module configured to connect the cognitive computation module with an Automated Guided Vehicle (AGV);
wherein the cognitive computation module communicates with the AGV to provide instructions for movement, enabling the humanoid robot to walk or drive itself to different locations autonomously based on analysed inputs and generated responses.

93. The system to create a humanoid robot comprising of claim 92, further comprising:
an interface module configured to connect the cognitive computation module with a Modular Mobility Platform (MMP);
wherein the cognitive computation module communicates with the MMP to provide instructions for movement, enabling the humanoid robot to walk or drive itself to different locations autonomously based on analysed inputs and generated responses.

94. The system to create a humanoid robot comprising of claim 92, further comprising:
wherein the MMP is equipped a legged walking mechanism for pedestrian-like mobility and or retractable wheels for efficient travel over smooth surfaces which enable the humanoid robot to navigate diverse terrains autonomously, adapting its mode of locomotion based on analysed inputs and generated responses to achieve optimal movement efficiency and versatility.

95. The system to create a humanoid robot comprising of claim 92, wherein the cognitive computation module is coupled/linked with at least one of the premises environment components like GPS, camera, speakerphone, microphone, Bluetooth devices, Wi-Fi devices, Cellular connected devices, or other sensors, RFID tags, ultrasonic sensors, or API connection with other IoT devices to access data of the premises.

96. The system to create a humanoid robot comprising of claim 92, wherein the Artificial intelligence module/humanoid robot module is coupled with the cognitive computation module with all possible hazardous situations, with all possible questions so that false positive situations are eliminated just by speaking and listening to answers to the questions in addition to using facial recognition data or camera input.

97. The system to create a humanoid robot comprising of claim 92, wherein one of the possible hazardous situations is warfare, fire, accident, space exploration, or any natural disasters.
